SEQUENCE LISTING (3 sequences)

SEQ ID NO: 1 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 308 aa, Homo sapiens
KQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSG

SEQ ID NO: 3 - protein, 308 aa, artificial sequence
  /note=“Description of artificial sequence kinase domain residues 117-424, mutations C218S/C276S/C296S”
KQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTSAEKLKKRMQGPIPERILGKMTVAIVKALYYLKEKHGVIHRDVKPSNILLDERGQIKLSDFGISGRLVDSKAKTRSAGSAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSG

(19) United States
(12) Patent Application Publication
(10) Pub. No.: US 2012/0107836 A1
(43) Pub. Date: May 3, 2012

(54) DEVELOPMENT OF FLUORESCENTLY P-LOOP LABELLED KINASES FOR SCREENING OF INHIBITORS

(76) Inventors: Daniel Rauh, Worms (DE); Jeffrey Raymond Simard, Salem, MA (US)

(21) Appl. No.: 13/272,709

(22) Filed: Oct. 13, 2011

Related U.S. Application Data
(63) Continuation-in-part of application No. PCT/EP2010/055129, filed on Apr. 19, 2010.
(60) Provisional application No. 61/170,375, filed on Apr. 17, 2009.

(30) Foreign Application Priority Data
Apr. 17, 2009 (EP) 09005492.5

(51) Int. Cl. C12Q 1/48; G01N 33/53; C12N 9/96

(52) U.S. Cl. 435/7.6; 435/188; 435/15

(57) ABSTRACT

The present invention relates to a kinase labelled at an amino acid naturally present or introduced in the P-loop of said kinase, wherein said labelling is effected at a free thiol or amino group of said amino acid and said label is (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase. The invention furthermore relates to a method of screening for kinase inhibitors, a method of determining the kinetics of ligand binding and/or of dissociation of a kinase inhibitor and a method of generating mutated kinases suitable for the screening of kinase inhibitors using the kinase of the present invention.

### RELATED APPLICATIONS AND INCORPORATION BY REFERENCE

This application is a continuation-in-part application of international patent application Serial No. PCT/EP2010/055129 filed 19 Apr. 2010, which published as PCT Publication No. WO 2010/119138 on 21 Oct. 2010, which claims benefit of European patent application Serial No. 09005492.5 filed 17 Apr. 2009 and U.S. provisional patent application Ser. No. 61/170,375 filed 17 Apr. 2009.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a kinase labelled at an amino acid naturally present or introduced in the P-loop of said kinase, wherein said labelling is effected at a free thiol or amino group of said amino acid and said label is (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase. The invention furthermore relates to a method of screening for kinase inhibitors, a method of determining the kinetics of ligand binding and/or dissociation of a kinase inhibitor and a method of generating mutated kinases suitable for screening of kinase inhibitors using the labelled kinase of the present invention.

### BACKGROUND OF THE INVENTION

Protein kinases are an important set of enzymes regulating key cellular processes. The improved understanding of aberrantly regulated kinase signaling in cancer biology (Gschwind and Fischer, 2004) has lead to the development of small organic molecules that are used to specifically target unwanted kinase activities and initiated the area of targeted cancer therapies (Zhang et al., 2009).

Most kinase inhibitors are Type I inhibitors such as dasatinib (Sprycel®), bind to the active “DFG-in” conformation of the kinase and compete with ATP in order to form critical hydrogen bonds with the kinase hinge region. In this conformation, the regulatory activation loop is open and extended, which allows ATP and substrates to bind (Knighton et al., 1991). The adenine of ATP forms a crucial hydrogen bond with the hinge region of the kinase—a short, flexible region located between the N- and C-terminal lobes of the kinase domains while the β and γ phosphates of ATP are coordinated by a complex network of ionic and hydrogen bonding interactions with several structural elements, including Mg²⁺ or Mn²⁺ ions, the Asp side chain of the conserved DFG motif, and amino acid residues in the glycine-rich loop located above the ATP binding cleft (Aimes et al., 2000).

However, the development of these types of inhibitors is challenged by an increasingly exhausted chemical space within the ATP binding site, poor inhibitor selectivity and efficacy as well as the emergence of drug resistance. Current medicinal chemistry research attempts to overcome these bottlenecks to develop effective long-term therapies by identifying and developing inhibitors that target alternative (i.e. allosteric) binding sites and/or stabilize inactive kinase conformations which are enzymatically incompetent (Zhang et al., 2009; Adrian et al., 2006; Calleja et al., 2009; Fischmann et al., 2009; Kirkland and McInnes, 2009).

One of these sites is only present in the inactive “DFG-out” kinase conformation and is moving to the forefront of kinase research. The DFG-out conformation results from structural changes in the activation loop induced by an 180° flip of the highly-conserved DFG motif (Liu and Gray, 2006; Pargellis et al., 2002), an event which also exposes a less-conserved allosteric site adjacent to the ATP binding site. Type II and Type III inhibitors bind to this less conserved allosteric site and are believed to have superior selectivity profiles, improved pharmacological properties (Copeland et al., 2006) and offer new opportunities for drug development (Liu and Gray, 2006).

More specifically, Type II inhibitors such as sorafenib (Nexavar®, Wan et al., 2004), imatinib (Gleevec®, Nagar et al., 2002) and BIRB-796, a selective inhibitor of p38α (Pargellis et al., 2002), bind to the hinge region and are ATP-competitive but extend into this allosteric site while Type III inhibitors bind exclusively within the allosteric pocket (Pargellis et al., 2002; Simard et al., submitted).

Until recently, approaches that allowed for the unambiguous identification of inhibitors which stabilize the inactive DFG-out conformation fell short or were not compatible with the high-throughput screening formats used by academia and industry to identify new hit compounds (Annis et al., 2004; Vogtherr et al., 2006), thus highlighting the need for innovative new approaches to detect and characterize such ligands.

The attachment of fluorophores to proteins is a well-established approach used to detect conformational changes in protein structure in response to ligand binding. In addition to the commercially-available probe acrylodan-labelled fatty acid binding protein (ADIFAB; Molecular Probes), which measures the concentration of unbound fatty acids in buffer (Richieri et al., 1999), this approach has been applied to various other proteins including acetylcholine binding protein (Hibbs et al., 2004), interleukin-1β (Yem et al., 1992) and various sugar and amino acid binding proteins (de Lorimier et al., 2002).

Citation or identification of any document in this application is not an admission that such document is available as prior art to the present invention.

### SUMMARY OF THE INVENTION

It would be desirable to have versatile means and methods for screening for specific kinase inhibitors. However, some kinases may be more or less sensitive to ligands which can influence or induce a DFG-in/out switch in conformation. Therefore, it would be useful to develop alternative screening strategies for sensitively detecting DFG-out binders, for kinases which readily adopt the DFG-out conformation as well as ligands that may bind within the ATP site and induce other conformational changes in target kinases which are not changes in the activation loop or DFG conformation. The solution to this technical problem is achieved by providing the embodiments characterized in the claims.

Accordingly, the present invention relates to a kinase labelled at an amino acid naturally present or introduced in the P-loop of said kinase, wherein said labelling is effected at a free thiol or amino group of said amino acid and said label may be (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase.

The present invention also relates to a method of generating a mutated kinase suitable for the screening of kinase inhibitors which may comprise: (a) replacing solvent exposed amino acids having a free thiol or amino group, if any, present in a kinase of interest outside the P-loop and/or amino acids having a free thiol or amino group at an unsuitable position within the P-loop with an amino acid not having a free thiol or amino group; (b) mutating an amino acid in the P-loop of said kinase of interest to an amino acid having a free thiol or amino group if no amino acid having a free thiol or amino group is present in the P-loop; (c) labelling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity of the kinase and/or does not interfere with the stability of the kinase; (d) contacting the kinase obtained in step (c) with a known inhibitor of said kinase; (e) recording the fluorescence emission signal at one or more wavelengths or a spectrum of said fluorescently labelled kinase of step (c) and (d) upon excitation; or (e)′ recording the EPR or NMR spectra of said spin-labelled kinase of step (c) and (d); and (f) comparing the fluorescence emission spectra recorded in step (e) or the EPR or NMR spectra recorded in step (e)′; wherein a difference in the fluorescence intensity at at least one wavelength, preferably at the emission maximum, and/or a shift in the fluorescence emission wavelength in the spectra of said fluorescently labelled kinase obtained in step (e), or an alteration in the EPR or NMR spectra of said spin-labelled or isotope-labelled kinase obtained in step (e)′ indicates that the kinase is suitable for the screening for kinase inhibitors.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Schematic representation of changes in P-loop and activation loop conformations triggered by ligand binding in p38α. a) Structurally important regions (P-loop; helix C; hinge region) of the active kinase domain (DFG-in) are labelled. b) Mobility of the activation loop in resonance to the activation loop. Type II/III inhibitors occupy a site that is present only in the DFG-out kinase conformation. This allosteric pocket is flanked by the DFG-motif and helix C. Type II/III inhibitor binding to the allosteric site causes a conformational change in the activation loop (black) and allows the P-loop (black) to adopt a more extended conformation. c) A Cys residue was mutated into the P-loop of p38α for subsequent labelling with an environmentally-sensitive fluorophore (large sphere) to generate a sensitive P-loop binding assay. Active (DFG-in) and inactive (DFG-out) kinase conformations are in equilibrium and result from structural changes in the activation loop. Structural changes of the activation loop are transmitted to the P-loop through a hydrophobic interface and change the chemical environment the fluorophore attached to the P-loop. Type I inhibitor (surface behind the large sphere in the left panel) binds to the hinge region of the active kinase (DFG-in) (left panel). In this particular case the P-loop folds over to directly interact with the inhibitor. In absence of ligands from active kinase (DFG-in) the P-loop adopts a more extended conformation (middle panel). Type II/III inhibitors (surface below large sphere in the right panel) bind to inactive (DFG-out) kinase conformations.

FIG. 2: Real-time and endpoint fluorescence measurements using ac-p38α labelled on the glycine-rich loop. Acrylodan emission at 475 nm decreases upon binding of BIRB-796 resulting in a red-shift (shift to longer wavelength) of the maximum emission wavelength in the bound state (A). Endpoint equilibrium measurements can be made to directly obtain the K_{d}. Ratiometric fluorescence data (R=512 nm/475 nm) were plotted on a logarithmic scale of inhibitor concentration to obtain the K_{d} (B). Ratiometric fluorescence data (R=445 nm/475 nm) can also be used to obtain the K_{d} (data not shown). Fluorescence traces can also be measured in real-time at a single wavelength (475 nm) to determine various kinetic rate constants. The fluorescence decay resulting from the addition of different amounts of BIRB-796 (large arrow) was fit (gray lines) to a first-order decay function to obtain k_{obs} (C). Experimentally determined k_{obs} values can then be plotted to determine kₒₙ for BIRB-796 any ligand. Extraction of BIRB-796 from ac-p38α using an excess of unlabelled p38α allowed direct determination of k_{off} which was also fit (gray lines) to a first-order function (D). The data presented above are representative of a typical set of experiments carried out for BIRB-796 using ac-p38α labelled on the glycine-rich loop.

FIG. 3. Real-time and endpoint fluorescence measurements of a Type III and Type I ligand using P-loop ac-p38α. Acrylodan emission at 475 nm decreases upon binding of the Type III ligand RL36 resulting in a red-shift of the maximum emission wavelength in the bound state (a). Similar but more intense changes were observed for SB203580, a Type I ligand known to stabilize the DFG-out conformation of p38α by interacting with the P-loop (b). Since Type III and Type II ligands (see FIG. 2) trigger a change in the activation loop conformation which results in an associated structural rearrangement of the glycine-rich loop (see FIG. 1a), both spectral shape and loss of intensity change similarly for both inhibitor types. Fluorescence traces were measured in real-time at a single wavelength (475 nm) to determine the rate of ligand binding and dissociation. The fluorescence decay resulting from the addition (black arrow) of 100 nM RL36 was fit (gray lines) to a first-order decay function to obtain k_{obs,on} ((a) center). Type I ligands such as SB203580 typically bind <5 sec ((b) center) and accurate curve fitting is not possible without the use of stop-flow fluorescence spectroscopy to increase the time resolution of the measurement. Extraction of each inhibitor from ac-p38α was accomplished by adding an excess of unlabelled p38α to the same sample (white arrow). Since it is known that the k_{off} is significantly slower than k_{obs,on} for all inhibitor types, it was possible to determine the k_{off} for each inhibitor by fitting (gray lines) the fluorescence increase to a first-order function. Ratiometric fluorescence data (R=512 nm/475 nm) were plotted on a logarithmic scale of inhibitor concentration to obtain the K_{d} for RL36 ((a) right) and SB203580 ((b) right). The Type II inhibitor imatinib does not bind to p38α and served as a negative control for RL36 ((a) right, black squares). The Type I inhibitor dasatinib binds to p38α but does not interact with the glycine-rich loop and served as a negative control for SB203580 ((b) right, black squares). The data presented above are representative of a typical set of experiments carried out using ac-p38α labelled on the glycine-rich loop.

FIG. 4: Fluorescence characterization and response of P-loop labelled p38α to different inhibitor types. The structures of various known inhibitor types (Type I, II or III) are shown in addition to the structures of Scios-469 and RL40, two hits identified in a compound screen. The P-loop was labelled by covalently modifying Y35C of p38α with the thiol-reactive fluorophore acrylodan and the changing fluorescence properties were examined upon binding of known DFG-out and DFG-in binders of p38α. All values for ΔRₘₐₓ and ΔI_{std} which meet the criteria deemed ideal fluorophore-protein conjugates (deLorimier et al. 2003) appear in bold text. In the case of traditional DFG-out binders (Type II and Type III inhibitors) or some Type I inhibitors which directly interact with the P-loop, acrylodan exhibits a large emission shift but there is an increase in emission at ˜512 nm relative to ˜475 nm, still allowing reliable binding curves to be measured despite the suboptimal ΔRₘₐₓ. However, superior ΔI_{std} values were obtained in the case of these same types of inhibitors. SB203580 is a Type I inhibitor of p38α known to stabilize the DFG-out conformation (as disclosed in EP 08 02 0341). Dasatinib binds to the hinge region of the kinase and does not interact with the DFG motif or the P-loop (Tokarski et al., 2006) and was not detected (ND) by this assay system. Type I inhibitors such as SB203580 and DFG-out binding Type II (BIRB-796) and III (RL36) inhibitors were sensitively detected, allowing for K_{d} and kinetic measurements. The kinetic measurements allow for the discrimination of Type I ligands, which bind very rapidly (<2 sec in this example) from Type II/III ligands, which are known to bind slowly to p38α (Pargellis et al. 2003). Two such ligands, RL40 and Scios-469, were detected in a screening initiative. Protein X-ray crystallography was later employed to understand the structural details behind the detection of these two ligands. [Note: * ΔI_{std} was calculated using emission intensities at 445 and 475 nm in presence and absence of ligand (R=445/475 nm is most optimal to detect ligand binding); ** ΔRmax was calculated using emission intensities at 475 and 512 nm in presence and absence of ligand (R=512/475 nm is most optimal to discriminate binding mode).]

FIG. 5. Crystal structures of RL40, Scios-469 and CP547632 confirm movement of the P-loop. The structure of RL40 in complex with p38α (a) reveals a unique and unexpected binding mode analogous to that observed in the structure of SB203580 reported previously (EP 08 02 0341) in which the ligand interacts with the P-loop by forming a unique π-π stacking with the Phe side chain of the DFG motif. The result of this interaction is the stabilization of the DFG-out conformation. An overlay of the structures for SB203580 and RL40 in p38α reveals that the aromatic cores of both inhibitors nicely overlay and form the same type of stacking interactions with the P-loop and activation-loop (b). Analogs of RL40 are typically observed binding to the hinge region of kinases and do not interact with the P-loop (Pierce et al., 2005), thus highlighting the benefit of using P-loop labelled kinases to enrich for ligands which take advantage of these unique binding modes. Additionally, the P-loop labelled kinase assay strongly detected the binding of Scios-469. Applicants co-crystallized Scios-469 with wild-type p38α and solved the structure to a resolution of 2.5 Å (c). Applicants observed a dramatic movement in the P-loop when compared to the apo structure of p38α. This movement is induced and stabilized by stacking interactions of the P-loop Tyr35 (the chosen labelling position for the assay) with hydrophobic features of the compound. This provides an example of how the P-loop labelled kinase assay can sensitively detect some Type I ligands which directly alter the conformation of the P-loop. (d) The carbonyl attached to the piperazine ring of Scios-469 forms two hydrogen bonds (dashed red lines) to the hinge region (pink) (backbone NH of Met109 and Gly110). The glycine-rich loop (green) folds over to directly interact with the inhibitor and shields the indole moiety and piperazine ring from the solvent. The DFG-motif (orange) is in the “in” conformation with Asp168 pointing into the ATP binding site. (e) Similar to Scios-469, the halogen substituted methoxybenzene of CP547632 bends over the gatekeeper (Thr106) and points into the hydrophobic subpocket. The carboxamide and the urea attached to the thiazole ring both form hydrogen bonds to the hinge region (backbone CO of His107, NH and CO of Met109). The pyrrolidine-butan moiety is kinked by 90° and points away from the solvent into the ATP pocket. The glycine-rich loop is less visible in the electron density and the DFG-motif is clearly in the “out” conformation.

FIG. 6. Kinetic and inhibitory characterization of wild type, unlabelled and acrylodan-labelled p38α. Note: The kinetic parameters were determined using the HTRF® assay from Cisbio and demonstrate that the introduced mutations (Cys119Ser/Cys162Ser/Tyr35Cys in p38α) do not significantly change the affinity of the kinase for ATP (ATP-Kₘ). Comparison of IC₅₀s, carried out at the Kₘ of each variant, show no significant effect of the mutations or labelling on the IC₅₀s of a few known Type I and II p38α inhibitors, thereby validating the chosen glycine-rich loop labelling site for the labelling approach of the invention. All reported values are the mean±s.d. of at least 3 independent experiments, each performed in duplicate.

FIG. 7. Time-dependency of K_{d} values for BIRB-796 (1) with p38α measured in a 384-well format. Binding curves for Type I inhibitors SB203580, Scios-469 and CP547632 as well as for the slow-binding Type II inhibitor BIRB-796 were obtained using p38α to demonstrate that inhibitor binding mode can be predicted in HTS formats in addition to measuring real-time kinetics of binding (see FIG. 2). For each ligand, ratiometric fluorescence (R=I_{λ512}/I_{λ475}) was measured over a range of concentrations at 5, 30, 90 and 300 min and plotted to determine the K_{d} of each ligand at each time point. The K_{d} of SB203580, Scios-469 and CP547632 did not change significantly after 5 min incubation with glycine-rich loop-labelled p38α at room temperature. The K_{d} of BIRB-796 decreased ˜3-fold over a period of 90 min. Incubation times of 90 min at room temperature were sufficient for Type II inhibitors to reach binding equilibrium with the kinase. K_{d} values determined in a 384-well format were 2 to 3-fold higher than when measured in the cuvette format (see Table 1), which is often attributable to higher DMSO concentrations and the addition of detergents for screening in HTS plates. All reported K_{d} values are the mean±s.d. of 4 independent experiments, each performed in triplicate.

FIG. 8. Real-time and endpoint fluorescence measurements of a Type II and Type I ligands using glycine-rich loop labelled MKK7. (A) The binding of K252a induces a decrease in fluorescence intensity of the labelled protein and a detectable change in the ratiometric emission at two wavelengths (R=472 nm/510 nm). (B) Using the endpoint methodology to directly determine K_{d}, the ratio of these emissions can be plotted against inhibitor concentration to obtain a K_{d} of 38 nM for K252a, which is in the correct range expected for these compounds (Karaman et al., 2008). As negative controls, sorafenib, a Type II inhibitor, which is not detected up to 10 uM was included. These findings are in line with expected results for MKK7, which shows an insensitivity to the DFG-out conformation and inhibitors which induce or stabilize the DFG-out conformation (Karaman et al., 2008). To demonstrate that the assay response is due to movement of the P-loop upon Type I inhibitor binding, dasatinib was also included as a negative control. Dasatinib is an ATP-competitive inhibitor or cSrc and Abl kinases and only inhibits MKK1 and MKK2 but with reported K_{d} values >1 uM (Karaman et al. 2008). Therefore, addition and detection of this Type I inhibitor was not expected for MKK7, which the data confirm (C). Real-time kinetic measurements and detection of binding and dissociation of K252a. As in FIG. 3 for p38α, the fluorescence change which occurs with binding is reversible upon addition of excess unlabelled MKK7 to extract the ligand from the labelled kinase. Since K252a is a Type I inhibitor, the kinetics of these processes are fast, as for the Type I inhibitor SB203580 of p38α shown in FIG. 3B.

### DETAILED DESCRIPTION OF THE INVENTION

The term “kinase” is well-known in the art and refers to a type of enzyme that transfers phosphate groups from high-energy donor molecules, such as ATP, to specific target molecules such as proteins. Kinases are classified under the enzyme commission (EC) number 2.7. According to the specificity, protein kinases can be subdivided into serine/threonine kinases (EC 2.7.11, e.g. p38α), tyrosine kinases (EC 2.7.10, e.g. the EGFR kinase domain), histidine kinases (EC 2.7.13), aspartic acid/glutamic acid kinases and mixed kinases (EC 2.7.12) which have more than one specificity (e.g. MEK being specific for serine/threonine and tyrosine).

Amino acids are defined as organic molecules that have a carboxylic and amino functional group. They are the essential building blocks of proteins. Examples of amino acids having a free thiol group are cysteine, belonging to the 20 proteinogenic amino acids, and acetyl-cysteine being a non-standard amino acid rarely occurring in natural amino acid sequences. Proteinogenic amino acids having a free amino group are lysine, histidine or arginine and amino acids being aromatic amines, such as tryptophan. Pyrrolysine, 5-hydroxylysine or o-aminotyrosine are non-standard amino acids having a free amino group. The amino acids asparagine and glutamine, although having a free amino group, are not suitable in the present invention as they are not reactive to labelling agents and are thus excluded.

Tryptophan is an aromatic amino acid having an amino group in its indole ring. Aromatic amines are weak bases and thus unprotonated at pH 7. However, they can still be modified using a highly reactive reagent such as an isothiocyanate, sulfonyl chloride or acid.

The kinase is labelled at a free thiol or amino group of an amino acid at the desired position in the kinase, i. e. in the P-loop. During labelling, the previously free thiol or amino group is involved in forming the covalent bond between the labelled amino acid and the label according to items (a) and (b).

Said amino acid to be labelled is located in the P-loop of the kinase. This means that only kinases having a P-loop or a structure equivalent thereto fall within the present invention. The P-loop (also called glycine-rich loop) is a highly flexible structural feature conserved among all ATP/GTP binding proteins (Saraste et al., 1990). In kinases, the P-loop contains the canonical Gly-X-Gly-X-X-Gly motif (where X is any amino acid) and is located in the N-terminal lobe of kinases where it serves as regulatory loop to guide the entry of ligands such as ATP into the ATP binding site of kinases (Wong et al., 2005).

Cysteines which are naturally present in a kinase of interest and are solvent-exposed can be located outside the P-loop or within the P-loop sequence. This equally applies to amino acids having a free amino group.

The modified kinase of the invention is labelled at an amino acid naturally present or introduced into the P-loop. If no suitable amino acid, i.e. one having a free thiol- or amino group, is present in the P-loop, said amino acid can be introduced, i.e. inserted by adding it or by replacing an existing amino acid, by techniques well-known in the art. In any case, it is to be understood for the avoidance of doubt that the amino acid is only labelled after its introduction into the P-loop if it is to be labelled by reaction with labelling reagents. The above techniques comprise site-directed mutagenesis as well as other recombinant, synthetic or semi-synthetic techniques. In case a non-standard amino acid is to be introduced into the kinase, an amino acid stretch containing said amino acid may be chemically synthesized and then connected to the remaining part(s) of the kinase which may have been produced recombinantly or synthetically. Alternatively, kinases expressed and designed to incorporate a specialized non-standard amino acid at the desired position for subsequent labelling may be produced recombinantly by applying an altered genetic code (see e. g. Liu and Schultz, 2010).

The process of labelling involves incubation of the kinase, e.g. the mutated kinase of the invention (e.g. the kinase with a cysteine introduced in the P-loop), with a thiol- or amino-reactive label under mild conditions resulting in the labelling of said mutated kinase at the desired position in the P-loop. In other words, it is in principle possible that only said desired position is labelled in the kinase which is a preferred embodiment, except for the labelling with a thiol-reactive spin-label, where alternatively the concomitant labelling with isotopes is envisaged (see below). Mild conditions refer to buffer pH (e.g. around pH7 for thiol-reactive probes), ratio of label to kinase, temperature and length of the incubation step (for thiol-reactive probes e.g. 4° C. and overnight in the dark) which are known to the skilled person and provided with instruction manuals of providers of thiol- and amino-reactive probes. Such conditions need to be optimized to slow down the reaction of the chosen thiol- or amino-reactive label to ensure that labelling of said kinase is specific to the desired labelling site. In the case of fluorophore labelling, it is necessary to carry out the incubation in the dark. Increased light exposure results in bleaching of the fluorophore and a less intense fluorescence emission. After labelling, the labelled kinase is preferably concentrated, purified by gel filtration experiments or washed several times with buffer to remove excess unreacted label. The wash buffer is typically the buffer used to store the labelled kinase and may also be the buffer in which the desired measurements are made.

The term “fluorophore” denotes a molecule or functional group within a molecule which absorbs energy such as a photon of a specific wavelength and emits energy, i.e. light at a different (but equally specific) wavelength (fluorescence) immediately upon absorbance (unlike the case in phosphorescence) without the involvement of a chemical reaction (as the case in bioluminescence). Usually the wavelength of the absorbed photon is in the ultraviolet range but can reach also into the infrared range. The wavelength of the emitted light is usually in the visible range. The amount and wavelength of the emitted energy depend primarily on the properties of the fluorophore but may also be influenced by the chemical environment surrounding the fluorophore. A number of fluorophores are sensitive to changes in their environment. This includes changes in the polarity, charge and/or in the conformation of the molecule they are attached to. Fluorescence occurs when a molecule relaxes to its ground state after being electrically excited which, for commonly used fluorescent compounds that emit photons with energies from the UV to near infrared, happens in the range of between 0.5 and 20 nanoseconds.

The term “thiol- or amino-reactive” denotes the property of a compound, e.g. a fluorophore, to specifically react with free thiol- or amino groups. This is due to a functional group present in said compound which directs a specific reaction with a thiol or amino group. These functional groups may be coupled to molecules such as fluorophores, spin labels or isotope-enriched molecules in order to provide specific labels attachable to free thiol- or amino-groups. Examples for thiol-specific compounds are e.g. haloalkyl compounds such as iodoacetamide, maleimides, Hg-Link™ phenylmercury compounds or TS-link™ reagents (both Invitrogen). Haloalkyl compounds react with thiol or amino-groups depending on the pH.

The term “spin label” (SL) denotes a molecule, generally an organic molecule, which possesses an unpaired electron, usually on a nitrogen atom, and has the ability to bind to another molecule. Spin labels are used as tools for probing proteins using EPR spectroscopy. The site-directed spin labelling (SDSL) technique allows one to monitor the conformation and dynamics of a protein. In such examinations, amino acid-specific SLs can be used.

Site-directed spin labelling is a technique for investigating protein local dynamics using electron spin resonance. SDSL is based on the specific reaction of spin labels with amino acids. A spin label built in protein structures can be detected by EPR spectroscopy. In SDSL, sites for attachment of spin labels such as thiol or amino groups, if not naturally present, are introduced into recombinantly expressed proteins by site-directed mutagenesis. In other words, by the above techniques, spin labels can be introduced into a kinase such that said kinase is specifically labelled only at the desired position. Functional groups contained within the spin label determine their specificity. At neutral pH, protein thiol groups specifically react with functional groups such as methanethiosulfonate, maleimide and iodoacetamide, creating a covalent bond with the amino acid cysteine. Spin labels are unique molecular reporters, in that they are paramagnetic, i.e. they contain an unpaired electron. Nitroxide spin labels are widely used for the study of macromolecular structure and dynamics because of their stability and simple EPR signal. The nitroxyl radical (N—O) is usually incorporated into a heterocyclic ring such as pyrrolidine, and the unpaired electron is predominantly localized to the N—O bond. Once incorporated into the protein, a spin label's motions are dictated by its local environment. Because spin labels are exquisitely sensitive to motion, this has profound effects on the EPR spectrum of the spin-label attached to the protein.

The signal arising from an unpaired electron can provide information about the motion, distance, and orientation of unpaired electrons in the sample with respect to each other and to the external magnetic field. For molecules free to move in solution, EPR works on a much faster time-scale than NMR (Nuclear Magnetic Resonance spectroscopy), and so can reveal details of much faster molecular motions, i.e. nanoseconds as opposed to microseconds for NMR. The gyromagnetic ratio of the electron is orders of magnitude larger than of nuclei commonly used in NMR, and so the technique is more sensitive, though it does require spin labelling.

The term “isotope” denotes a chemical species of a chemical element having different atomic mass (mass number) than the most abundant species of said element. Isotopes of an element have nuclei with the same number of protons (the same atomic number) but different numbers of neutrons.

Isotopes suitable for EPR or NMR need to have a nonzero nuclear spin. The most common isotopes currently used are ¹H, ²D, ¹⁵N, ¹³C, and ³¹P.

Whereas also a thiol-reactive spin-label alone at a specific position in the P-loop of a kinase can be used in the present invention, it is preferred that a kinase specifically labelled with a thiol-reactive spin label in the P-loop is also labelled with an isotope (as described in detail further below). However, if only isotope-labelling is used, it is preferred that the isotope is only present at the specific desired position in the P-loop of the kinase and that no other positions in the kinase are thereby labelled.

The term “isotope-enriched” denotes that a compound, e.g. a thiol- or amino-reactive label has been synthesized using or reacted with an isotope so that said isotope is introduced into said compound. The compound may comprise one or more isotopes of one or more different species. Regarding the position and number of labels, the same applies as described above for isotopes.

The label has to be positioned so that it does not significantly disrupt or inhibit the kinase's catalytic activity and does not interfere with its stability. In principle, the assay of the invention does not rely on the measurement of the catalytic activity of the labelled kinase of the invention and, therefore, does not require prior knowledge of the substrate of the kinase. However, it is preferable that essentially no interference with the catalytic activity takes place to allow for the reasonable comparison of the binding activity of potential inhibitors to the labelled kinase of the invention with the wild-type kinase from which it is derived. In the case of a kinase that is isotopically labelled on an amino acid, e.g. a cysteine, and produced by growing host organisms expressing the kinase with isotopically labelled amino acid already incorporated into the sequence, inhibition of the activity or interference with the stability of the kinase is unlikely. On the other hand, care also, has to be taken when selecting the position in the P-loop where the label is to be introduced. If no suitable amino acid is present at the position of choice, the amino acid present at said position must be replaced with an amino acid containing a free thiol or amino group other than the α-amino group involved in peptide bonds. The P-loop confers ATPase activity to the kinase. Accordingly, a suitable labelling position should be chosen such that the kinase retains at least 70%, preferably at least 80%, more preferably at least 90%, and most preferably 100% of its ATPase activity. Tests of how to evaluate the activity and stability of a kinase prior to and after replacement of an amino acid are well known to the skilled person and include visual inspection of the purified protein, circular dichroism (CD) spectroscopy, crystallization and structure determination, enzyme activity assays, protein melting curves, differential scanning calorimetry and NMR spectroscopy.

As described above, the P-loop comprises a highly conserved glycine-rich motif G-X-G-X-X-G, also called the ATP/GTP phosphate binding motif in ATPases/GTPases, respectively. The conserved glycines are suggested to be critical for optimal positioning of the phosphates of ATP or GTP for efficient phospho transfer to the docked substrate of the enzyme. Although, in principle, any amino acid within said conserved motif could be chosen for replacement and/or labelling according to the invention, it is preferred that a less-conserved amino acid at the variable positions in the glycine-rich motif (designated as x) is chosen. Choosing one of the conserved glycine residues might interfere with the ATPase activity of the kinase which should preferably be avoided in order to obtain a labelled kinase with at least similar, preferably essentially unaltered catalytic activity as compared to the naturally occurring kinase as also described above. It is further preferred that the amino acid at a position X to be replaced is an aromatic amino acid such as phenylalanine or a tyrosine. Both phenylalanine and tyrosine are bulky and are expected to adopt similar conformational rearrangements with ligand binding when compared to the covalently attached labels according to the invention, in particular the thiol-reactive label acrylodan.

In this regard, no inhibition of the catalytic activity is present if at least 90% of the catalytic activity of the kinase, preferably the wild-type kinase in its active state, are retained, preferably at least 95%, more preferably at least 98%. Most preferably, the catalytic activity of the kinase is fully retained. The term “does not inhibit the catalytic activity” is thus, in some embodiments where the catalytic activity amounts to less than 100%, to be equated with and having the meaning of “does not essentially interfere with the catalytic activity”. The catalytic activity can indirectly be determined by comparing the IC50 value of an inhibitor in the labelled kinase of the invention and the unlabelled kinase from which it is derived (using an amount of ATP equal to the ATP-Km). If the IC50 values are within the same range, i.e. if they do not differ by more than a factor of 5, this indicates that the catalytic activity is essentially the same (and that the modifications to the kinase did not alter inhibitor affinity for the kinase). It is preferred that the labelled kinase of the invention and the unlabelled kinase differ by not more than the factor 4, more preferably by not more than the factor 3, even more preferably by not more than the factor 2. The skilled person is aware that a difference between both IC50 values of up to the factor 5 is well within the usual variance associated with these measurements. Such IC50 values ensure that the catalytic activity of both kinases is essentially the same. Regarding stability, the amino acid introduced does not interfere with the essential intramolecular contacts that ensure structural stability of the protein, so that the kinase can carry out the biological function described herein.

To overcome the drawbacks of presently existing screening methods, the present invention involves a labelling strategy to create e.g. fluorescent-tagged kinases which (i) are highly sensitive to the binding of kinase inhibitors, (ii) can be used to measure the kinetics of ligand binding and dissociation in real-time, (iii) can be used to directly measure the Kd of these ligands and (iv) is rapid, robust, reproducible and adaptable to high-throughput screening methods.

In contrast to the prior art and as demonstrated in the appended examples, the present invention provides kinases and screening methods using these kinases which enables for screening for inhibitors with a reduced effort and material and as well as a superior reliability. This is essentially achieved by providing a labelling strategy for a kinase such that the label alters its behaviour in reaction to changes in its environment caused e.g. by conformational changes in the P-loop of the kinase.

Besides conventional kinase assays for the screening of modulators of kinase activity, various approaches have recently been developed. However, many of these approaches suffer from major drawbacks. For example, Annis et al. (2004) describe an approach using affinity selection-mass spectrometry (AS-MS). This method is described as suitable for high-throughput screening. However, a size exclusion chromatography step has to be applied prior to the examination of each probe which is time-consuming and requires a lot of material.

De Lorimier et al. (2002) describe a family of biosensors based on bacterial proteins binding to small molecule ligands which were modified and labelled with different environmentally sensitive fluorophores. Upon ligand binding, the fluorophores alter their emission wavelength and/or intensity thus indicating the presence and/or concentration of the specific ligand bound to a probe. However, the labelling of kinases and the use of said kinases in the screening for specific inhibitors is neither disclosed nor suggested.

More recently, two additional binding assays based on the displacement of prebound probes from p38α kinase were also reported: one made use of a fluorophore-labelled inhibitor (Tecle et al., 2009) and the other employed an enzyme fragment complementation-based approach (Kluter et al., 2009). In the latter case, a chemiluminescence read-out was generated by the displacement of a prebound inhibitor-peptide probe, which then complements and activates β-galactosidase to catalyze a chemiluminescence reaction that serves as the assay read-out. Although these approaches were demonstrated to be suitable for determining the affinities of displacing ligands using end point measurements, analysis of kinetic parameters (kₒₙ and k_{off}) is less straightforward since signal detection is rate-limited by the well-characterized slow dissociation of the chosen pyrazolourea-based probes from p38α (Pargellis et al., 2002).

The principle underlying the present invention is that the P-loop reacts to conformational changes of the activation loop upon binding of a type II or type III inhibitor. The activation loop is a flexible segment near the entrance to the active site which forms the substrate binding cleft of most kinases and can be phosphorylated on one or more amino acids to provide an important regulatory mechanism throughout the protein kinase superfamily (Johnson and Lewis, 2001; Taylor and Radzio-Andzelm, 1994; Johnson et al., 1996). The activation loop consists of several amino acids which form a loop that is flexible in most kinases which begins with a highly-conserved aspartate-phenylalanine-glycine (DFG) motif in the ATP binding site and extends out between the N- and C-lobes of the kinase. The activation loop is a structural component crucial for enzymatic kinase activity. It is part of the substrate binding cleft and contains several amino acid residues which assist in the recognition of specific substrates and also contains serines, threonines or tyrosines which can be phosphorylated. The conformation of the activation loop is believed to be in dynamic equilibrium between the DFG-in (active kinase) and DFG-out (inactive kinase) conformations. Phosphorylation and/or binding of interaction partners (other proteins or DNA) result in a shift of the equilibrium. In the DFG-in conformation, the aspartate contained in the motif is pointed into the ATP binding site and the adjacent phenylalanine is pointed away from the ATP site and into the an adjacent allosteric site. When the conserved DFG motif forming part of the activation loop adopts the in-conformation, ATP-competitive inhibitors (Type I inhibitors) can bind to the kinase. In the DFG-out conformation, the positions of these residues are flipped 180° in orientation. The out-conformation of the activation loop prevents ATP and substrate binding.

Besides controlling the entry of ligands and substrates into the ATP binding sites as described above, the P-loop helps to shield ATP and other ligands from the surrounding solvent. It has been shown to adopt various conformations related to the binding of some Type I inhibitors in the ATP binding pocket (Hanks and Hunter, 1995 (Hanks and Hunter, 1995; Mapelli et al., 2005).

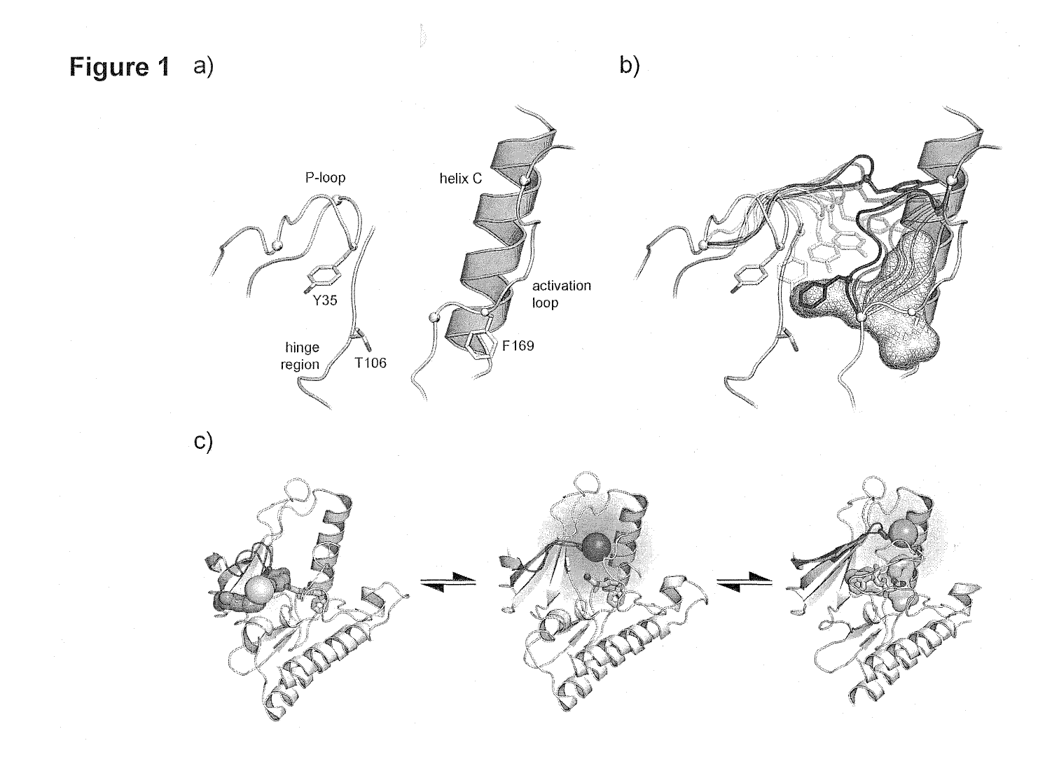

In accordance with the present invention, allosteric inhibitors (see FIG. 1c) were detected using a fluorescent- or spin-labelled P-loop assay system. The attached fluorophore or spin label reports movements in the P-loop which occur when the activation loop of the kinase adopts the DFG-out conformation. As shown in the appended example, the introduction of a Cys residue via site-directed mutagenesis into the position directly preceding the third Gly of the Gly-X-Gly-X-X-Gly motif to specifically label the P-loop with the environmentally-sensitive fluorophore acrylodan results in a kinase having the ability to aid in screening for inhibitors. The residue at this site is conserved as a Tyr or Phe in approximately 80% of all human kinases, suggesting a role for the aromatic ring system of these side chains in mediating the cross-talk of this loop with other structural features and ligands.

More importantly, this observation suggested that introduction of the planar ring system of acrylodan would be well tolerated by the kinase.

The present inventors recently developed a robust assay system in which they tagged the activation loop of target kinases (co-pending applications EP 08 01 3340 and EP 08 02 0341), allowing for the direct measurement of the dissociation constant (K_{d}), rate constant (kₒₙ) and rate constant of dissociation (k_{off}) of various ligands, allowing for the first time Type III ligands of cSrc and p38α to be identified and which led to the development of potent Type II inhibitors of gatekeeper mutated drug resistant cSrc-T338M. Furthermore, a new Type III binding mode for the thiazole-urea scaffold in p38α and several unique Type I ligands could be identified that stabilize the DFG-out conformation of p38α. The sensitivity in detecting ligands that stabilize the DFG-out conformation is significantly enhanced by using this approach to screen compound libraries since it utilizes the unphosphorylated inactive form of the kinase, which favours adoption of the DFG-out conformation. These earlier studies highlight the far-reaching implications of assays which can be used to screen for and enrich these types of ligands. However, in order to avoid potential changes in kinase activity resulting from alterations in the DFG-in/out conformational equilibrium or significant changes in the affinity of known inhibitors of the target kinase upon labelling of the activation loop, the alternative labelling strategy for identifying and characterizing Type II and Type III inhibitors as provided by the present invention makes said changes in activity less likely.

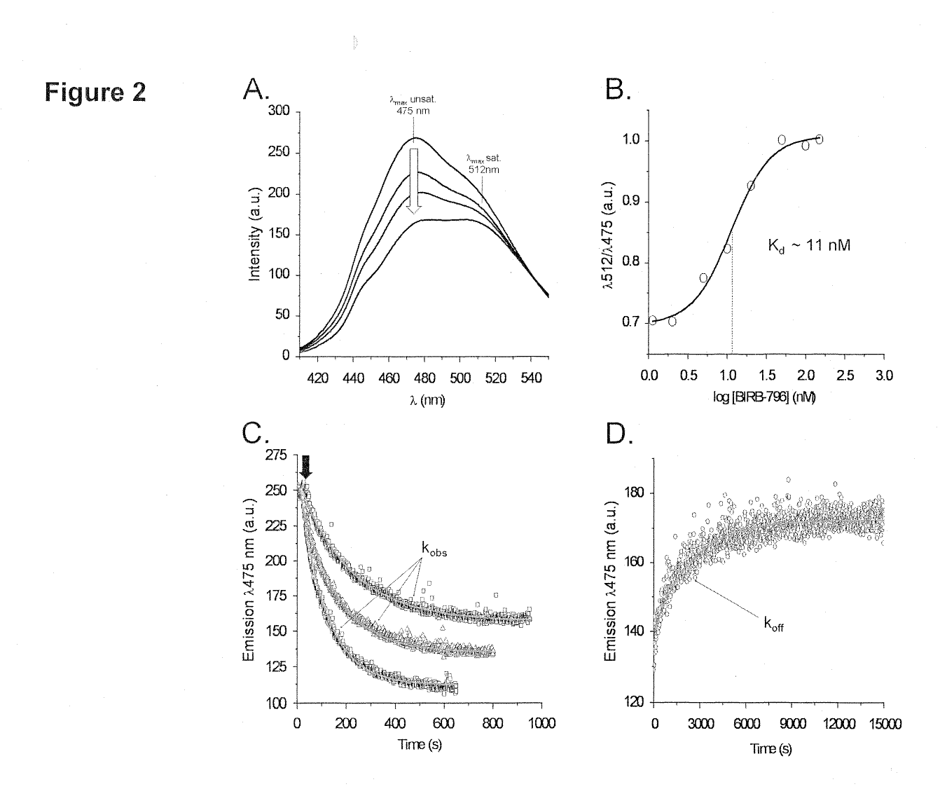

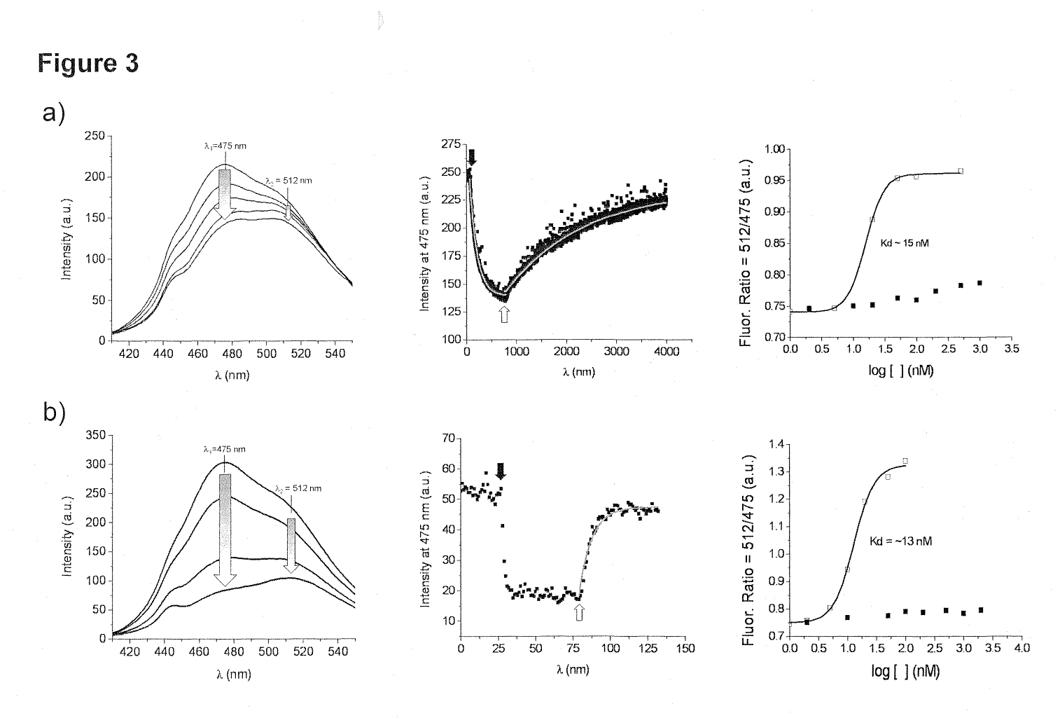

As shown in the appended examples, the present invention demonstrates the ability of P-loop labelled kinases to sensitively detect the binding of inhibitors with different binding modes, such as Type II and Type III inhibitors which induce changes in the environment of the fluorophore, e. g. a conformational change in the P-loop via movement of the activation loop to the DFG-out conformation, and alters its fluorescence properties (see FIG. 2a). Type II and Type III inhibitors are easily discriminated in HTS formats by monitoring time-dependent changes in fluorescence signal or K_{d} over time, or in cuvettes by measuring kₒₙ (<5 s for Type I binders). The present assay is also able to strongly detect Type I ligands which stabilize the DFG-out conformation by way of a unique binding mode. Such ligands bind within the ATP-binding site but utilize a unique ring-stacking interaction which forms between the inhibitor molecule, the highly-conserved Phe of the DFG motif and the planar ring system of the residue typically found at the chosen labelling position in the P-loop (Tyr35 in p38α). Lastly, some Type I inhibitors which bind to the DFG-in conformation have been shown to directly interact with the described Tyr/Phe side chain of the P-loop (Tamayo et al. 2005). By using this position to label the kinase, the detection of these types of inhibitors is also possible (FIG. 3B—right panel), without inducing the DFG-out conformation or movement of the activation loop. In comparison to the recently reported assay in which the activation loop is directly labelled with a fluorophore (patent applications EP 08 01 3340 and EP 08 02 0341), this assay system also utilizes the unphosphorylated form of the kinase and provides a powerful alternative screening tool for detecting changes in the activation loop conformation correlated with ligand binding, such as that induced by Type II and Type III inhibitors. Moreover, the druggability of the allosteric pocket likely varies between kinases and will be sensitive to the ability of the kinase to adopt the DFG-out conformation, thereby making the present invention an attractive alternative approach for detecting and designing high affinity Type I compounds which interact directly with the P-loop and induce conformational changes therein. The benefits of the identification of such Type I ligands should not be, underestimated since they might qualify as starting points for further development into Type II inhibitors that extend in the direction of the less conserved allosteric site (Liu and Gray, 2006).

Some Type I inhibitors also stabilize the DFG-out conformation. The key to being able to detect Type I DFG-out binders using the present invention is the ability to perform screens using the unphosphorylated form of the kinase in the absence of both substrate and ATP. As mentioned above, the unphosphorylated form of the kinase is more likely to adopt the DFG-out conformation in which residues in the DFG motif or N-terminal regions of the activation loop can interact with the ligand and thus enhance affinity by flipping into the ATP site to contact the ATP-competitive ligand. This is in contrast to classical activity-based assays that require the phosphorylated kinase, which is more likely to be found in the DFG-in conformation, thereby lowering the affinities of DFG-out binders and making it less likely that such preferred hits are detected (Seeliger et al., 2007). The established use of traditional activity-based assays in screening campaigns desensitizes the detection of DFG-out binders and could e. g. explain the lack of information in the literature about the binding of the VEGFR2 inhibitor CP547632 to active (i.e., phosphorylated) kinases other than VEGFR2. The reported high specificity of CP547632 has led to its application as a VEGFR2 inhibitor in clinical trials to stop tumour growth and proliferation by inhibiting angiogenesis. Given the submicromolar affinities of CP547632 detected using unphosphorylated p38α with the approach of the present invention, these findings could also stimulate further studies of this clinically relevant compound or close derivatives for the treatment of other kinase-associated diseases. Kinases exist in both phosphorylated and unphosphorylated forms inside the cell and the relative abundance of these species regulates kinase activity and signaling pathways. Thus, unphosphorylated kinases also represent biologically relevant and attractive drug targets. Additionally, the structural information provided here for CP547632 in complex with p38α (i.e., new type of hinge contact) could stimulate further medicinal chemistry efforts to build on the affine portions of this molecule to extend into the adjacent allosteric site and generate more pharmacologically desirable Type II inhibitors that bind to inactive kinase conformations.

By labelling the glycine-rich loop, not only is the goal of identifying DFG-out binders in applicable kinases achieved but it also allows the detection of Type I ligands that gain affinity for the DFG-in conformation by directly inducing conformational changes in the glycine-rich loop of kinases. This feature is a further advantage of the approach of the present invention over previous assays. By using glycine-rich loop labelled p38α as an exemplary kinase, Type I inhibitors such as Scios-469 were sensitively detected, which bind to the DFG-in conformation of p38α. Such compounds gain affinity for the kinase by inducing changes in the conformation of the glycine-rich loop that help shield the ligand from the surrounding solvent (Hanks and Hunter, 1995; Mapelli et al., 2005; Patel et al., 2009). Since the position in the glycine-rich loop often, but not always, responsible for these interactions is conserved as an aromatic Tyr or Phe in more than 80% of kinases, the present invention extends existing screening assays to additional kinases, including many kinases that are not regulated by a readily inducible DFG-in/out equilibrium. Detection of Type I inhibitors that interact with the glycine-rich loop may provide insights for the development of new scaffolds that take advantage of these interactions while avoiding the more traditional focus on identifying new types of hinge region contacts. Changes in glycine-rich loop conformation may also provide additional ways of improving Type I inhibitor specificities.

In a preferred embodiment, the kinase is a serine/threonine kinase or a tyrosine kinase.

In another preferred embodiment, the kinase is a MEK kinase, CSK, an Aurora kinase, GSK-3β, cSrc, EGFR, Abl, DDR1, LCK, a CDK, p38α or another MAPK.

Mitogen-activated protein (MAP) kinases (EC 2.7.11.24) are serine/threonine-specific protein kinases that respond to extracellular stimuli (mitogens) and regulate various cellular activities, such as gene expression, mitosis, differentiation, and cell survival/apoptosis. Extracellular stimuli lead to activation of a MAP kinase via a signaling cascade (“MAPK cascade”) composed of a MAP kinase, MAP kinase kinase (MKK or MAP2K) and MAP kinase kinase kinase (MKKK or MAP3K, EC 2.7.11.25).

A MAP3K that is activated by extracellular stimuli phosphorylates a MAP2K on its serine and/or threonine residues, and then this MAP2K activates a MAP kinase through phosphorylation on its serine and/or tyrosine residues. This MAP kinase signaling cascade has been evolutionarily well-conserved from yeast to mammals.

To date, six distinct groups of MAPKs have been characterized in mammals:
1. extracellular signal-regulated kinases (ERK1, ERK2). The ERK (also known as classical MAP kinases) signaling pathway is preferentially activated in response to growth factors and phorbol ester (a tumor promoter), and regulates cell proliferation and cell differentiation.
2. c-Jun N-terminal kinases (JNKs), (MAPK8, MAPK9, MAPK10), also known as stress-activated protein kinases (SAPKs).
3. p38 isoforms are p38α (MAPK14), p38β (MAPK11), p38γ (MAPK12 or ERK6) and p38δ (MAPK13 or SAPK4). Both JNK and p38 signaling pathways are responsive to stress stimuli, such as cytokines, ultraviolet irradiation, heat shock, and osmotic shock, and are involved in cell differentiation and apoptosis. p38α MAP Kinase (MAPK), also called RK or CSBP, is the mammalian orthologue of the yeast HOG kinase which participates in a signaling cascade controlling cellular responses to cytokines and stress. Similar to the SAPK/JNK pathway, p38 MAP kinase is activated by a variety of cellular stresses including osmotic shock, inflammatory cytokines, lipopolysaccharides (LPS), ultraviolet light and growth factors. p38 MAP kinase is activated by phosphorylation at Thr180 and Tyr182.
4. ERK5 (MAPK7), which has been found recently, is activated both by growth factors and by stress stimuli, and it participates in cell proliferation.
5. ERK3 (MAPK6) and ERK4 (MAPK4) are structurally related atypical MAPKs which possess an SEG (serine-glutamic acid-glycine) motif in the activation loop and display major differences only in the C-terminal extension.
6. ERK7/8 (MAPK15) are the most recently discovered members of the MAPK family and behave similar to ERK3/4.

Mitogen-activated protein kinase kinase forms a family of kinases which phosphorylates mitogen-activated protein kinase. They are also known as MAP2K and classified as EC 2.7.12.2. Seven genes exist. These encode MAP2K1 (MEK1), MAP2K2 (MEK2), MAP2K3 (MKK3), MAP2K4 (MKK4), MAP2K5 (MKK5), MAP2K6 (aka MKK6), MAP2K7 (MKK7). The activators of p38 (MKK3 and MKK4), JNK (MKK4), and ERK (MEK1 and MEK2) define independent MAP kinase signal transduction pathways.

Aurora kinases A (also known as Aurora, Aurora-2, AIK, AIR-1, AIRK1, AYK1, BTAK, Eg2, MmIAK1, ARK1 and STK15), B (also known as Aurora-1, AIM-1, AIK2, AIR-2, AIRK-2, ARK2, IAL-1 and STK12) and C (also known as AIK3) participate in several biological processes, including cytokinesis and dysregulated chromosome segregation. These important regulators of mitosis are over-expressed in diverse solid tumors. One member of this family of serine/threonine kinases, human Aurora A, has been proposed as a drug target in pancreatic cancer. The recent determination of the three-dimensional structure of Aurora A has shown that Aurora kinases exhibit unique conformations around the activation loop region. This property has boosted the search and development of inhibitors of Aurora kinases, which might also function as novel anti-oncogenic agents.

Glycogen synthase kinase 3 (GSK-3) is a serine/threonine protein kinase which in addition to the serine/threonine kinase activity has the unique ability to auto-phosphorylate on tyrosine residues. The phosphorylation of target proteins by GSK-3 usually inhibits their activity (as in the case of glycogen synthase and NFAT). GSK-3 is unusual among the kinases in that it usually requires a “priming kinase” to first phosphorylate a target protein and only then can GSK-3 additionally phosphorylate the target protein. In mammals GSK-3 is encoded by two known genes, GSK-3 alpha and beta. Aside from roles in pattern formation and cell proliferation during embryonic development, there is recent evidence for a role in tumor formation via regulation of cell division and apoptosis. Human glycogen synthase kinase-3 beta (GSK3β) is also associated with several pathophysiological conditions such as obesity, diabetes, Alzheimer's disease and bipolar disorder.

The Src family of proto-oncogenic tyrosine kinases transmit integrin-dependent signals central to cell movement and proliferation. The Src family includes nine members: Src, Lck, Hck, Fyn, Blk, Lyn, Fgr, Yes, and Yrk. These kinases have been instrumental to the modern understanding of cancer as a disease with disregulated cell growth and division. The cSrc proto-oncogene codes for the cSrc tyrosine kinase. Besides its kinase domain, cSrc is further comprised of an SH2 domain and an SH3 domain, which act as adaptor proteins for the formation of multi-enzyme complexes with the Src kinase domain. These domains are also involved in the auto-inhibition of the cSrc kinase domain. Mutations in this gene could be involved in the malignant progression of cancer cells. This protein specifically phosphorylates Tyr-504 residue on human leukocyte-specific protein tyrosine kinase (Lck), which acts as a negative regulatory site. It may also act on the Lyn and Fyn kinases.

Leukocyte-specific protein tyrosine kinase (Lck) is a protein that is found inside lymphocytes such as T-cells. Lck is a tyrosine kinase which phosphorylates tyrosine residues of certain proteins involved in the intracellular signaling pathways of lymphocytes. The N-terminal tail of Lck is myristoylated and palmitoylated, which tethers the protein to the plasma membrane of the cell. The protein furthermore contains an SH3 domain, an SH2 domain and in the C-terminal part the tyrosine kinase domain. The tyrosine phosphorylation cascade initiated by Lck culminates in the intracellular mobilization of calcium (Ca²⁺) ions and activation of important signaling cascades within the lymphocyte. These include the Ras-MEK-ERK pathway, which goes on to activate certain transcription factors such as NFAT, NFκB, and AP-1 which then regulate the production of a plethora of gene products, most notably, cytokines such as Interleukin-2 that promote long-term proliferation and differentiation of the activated lymphocytes. Aberrant expression of Lck has been associated with thymic tumors, T-cell leukemia and colon cancers.

The catalytic activity of the Src family of tyrosine kinases is suppressed by phosphorylation on a tyrosine residue located near the C terminus (Tyr 527 in cSrc), which is catalyzed by C-terminal Src Kinase (Csk). Given the promiscuity of most tyrosine kinases, it is remarkable that the C-terminal tails of the Src family kinases are the only known targets of Csk. Interactions between Csk and cSrc, most likely representative for Src kinases, position the C-terminal tail of cSrc at the edge of the active site of Csk. Csk cannot phosphorylate substrates that lack this docking mechanism because the conventional substrate binding site used by most tyrosine kinases to recognize substrates is destabilized in Csk by a deletion in the activation loop (Levinson, 2008).

The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Active EGFR occurs as a dimer. EGFR dimerization is induced by ligand binding to the extracellular receptor domain and stimulates its intrinsic intracellular protein-tyrosine kinase activity. As a result, autophosphorylation of several tyrosine residues in the C-terminal (intracellular) domain of EGFR occurs. This autophosphorylation elicits downstream activation and signaling by several other proteins that associate with the phosphorylated tyrosines through their own phosphotyrosine-binding SH2 domains. The kinase domain of EGFR can also cross-phosphorylate tyrosine residues of other receptors it is aggregated with, and can itself be activated in that manner. The EGFR signaling cascade activates several downstream signaling proteins which then initiate several signal transduction cascades, principally the MAPK, Akt and JNK pathways, leading to DNA synthesis and cell proliferation. Such pathways modulate phenotypes such as cell migration, adhesion, and proliferation. Mutations that lead to EGFR overexpression (known as upregulation) or overactivity have been associated with a number of cancers. Consequently, mutations of EGFR have been identified in several types of cancer, and it is the target of an expanding class of anticancer therapies.

The ABL1-protooncogene encodes a cytoplasmic and nuclear protein tyrosine kinase that has been implicated in processes of cell differentiation, cell division, cell adhesion and stress response. The activity of c-Abl protein is negatively regulated by its SH3 domain. A genetic deletion of the SH3 domain turns ABL1 into an oncogene. This genetic deletion, caused by the (9; 22) gene translocation results in the head-to-tail fusion of the BCR (MIM:151410) and ABL1 genes present in many cases of chronic myelogeneous leukemia. The DNA-binding activity of the ubiquitously expressed ABL1 tyrosine kinase is regulated by CDC2-mediated phosphorylation, suggesting a cell cycle function for ABL1.

Discoidin domain receptor family, member 1, also known as DDR1 or CD167a (cluster of differentiation 167a), is a receptor tyrosine kinase (RTK) that is widely expressed in normal and transformed epithelial cells and is activated by various types of collagen. This protein belongs to a subfamily of tyrosine kinase receptors with a homology region similar to the Dictyostelium discoideum protein discoidin I in their extracellular domain. Its autophosphorylation is achieved by all collagens so far tested (type I to type VI). In situ studies and Northern-blot analysis showed that expression of this encoded protein is restricted to epithelial cells, particularly in the kidney, lung, gastrointestinal tract, and brain. In addition, this protein is significantly over-expressed in several human tumors from breast, ovarian, esophageal, and pediatric brain.

The kinases described above are preferred embodiments because all of them are involved in the development of diseases such as cancer for which at present not suitable cure is available or an improved treatment regimen is desired.

Using p38α, a kinase for which structural information was available, the present inventors demonstrated the applicability of the labelled kinase of the invention for screening purposes. Unexpectedly, the kinase could be prepared for labelling with a minimum of effort but also the labelled kinase exerted the desired properties, i.e. the introduced label proved suitable for the detection of conformational changes induced by binding of a specific inhibitor, in this case the known inhibitor BIRB-796 and several smaller BIRB-796 analogs.

A further kinase which, in its labelled form according to the invention, can be applied for screening for specific inhibitors is MKK7 (reviewed e. g. in Wang et al., 2007). Both Type I and Type II inhibitors for MKK7 were analysed and their pharmacological profile could be refined to obtain more potent inhibitors, as detailed in example 7.

In another preferred embodiment, the amino acid labelled is cysteine, lysine, arginine or histidine.

Cysteine has a free thiol group, whereas lysine, arginine or histidine each possess at least one free amino group.

In another preferred embodiment, one or more solvent-exposed cysteines present outside the P-loop are deleted or replaced.

If more than one amino acid having a free thiol or amino group is present in a kinase of interest prior to labelling, specific labelling of the amino acid in the P-loop may not be possible. Therefore, as discussed above, amino acids present in the kinase and having a free thiol or amino group should be deleted or replaced with another amino acid not having a free thiol or amino group if they are predicted or shown to be solvent-exposed. Cysteines which are naturally present in a kinase of interest and are solvent-exposed can be located outside the P-loop, in which case they should be deleted or replaced with another amino acid not having a free thiol group. This equally applies to amino acids having a free amino group which should then be replaced with an amino acid not having a reactive free amino group. In case that one or more amino acids having a free amino group is already present in the P-loop, amino acids having a free amino group and present in the P-loop in addition to the amino acid to be labelled, should be replaced or deleted, whichever of these mutations to the kinase does not inhibit its catalytic activity or interfere with its stability. In summary, said mutations result in a kinase which is specifically labelled at the desired position in the P-loop.

The term “solvent-exposed” refers to the position of an amino acid in the context of the three dimensional structure of the protein of which it is a part. Amino acids buried within the protein body are completely surrounded by other amino acids and thus do not have any contact with the solvent. In contrast, solvent-exposed amino acids are partially or fully exposed to the surrounding solvent and are thus accessible to chemicals potentially able to modify them. This applies e.g. to thiol- or amino-reactive labels used in the present invention which can react with solvent-exposed amino acids having a free thiol- or amino-group.

The term “delete” refers to excision of an amino acid without replacing it with another amino acid whereas the term “replace” refers to the substitution of an amino acid with another amino acid. If an amino acid is replaced with another amino acid or deleted, the amino acid to be replaced or to be deleted is preferably chosen such that the amino acid deleted or replaced does not result in a kinase with inhibited catalytic activity and does not interfere with the stability of the resulting kinase.

In a more preferred embodiment, the kinase is p38α and a cysteine to be labelled is introduced at position 35 of SEQ ID NO: 1 and preferably the cysteines at positions 119 and 162 of SEQ ID NO: 1 are replaced with another amino acid not having a free thiol group such as serine.

In an alternative more preferred embodiment, the kinase is MKK7 and a cysteine to be labelled is naturally present in the P-loop at position 147 (position 31 in SEQ ID NO:2 corresponding to the kinase domain of MKK7) and preferably cysteines at positions 218, 276 and 296 (positions 102, 160 and 180 of SEQ ID NO: 2) are replaced with another amino acid not having a free thiol group such as serine. The MKK7 kinase domain having the cysteines at positions 218, 276 and 296 mutated to serines is depicted in SEQ ID NO: 3.

In general, amino acid replacements should be conservative. For cysteine, this means that it is preferably replaced with serine. In general, replacements of amino acids with different amino acids may be evaluated in view of whether they are conservative using the PAM250 Scoring matrix. The matrix is frequently used to score aligned peptide sequences to determine the similarity of those sequences (Pearson, 1990).

As described above, if not naturally present, an amino acid having a free thiol- or amino group has to be introduced into the P-loop of a kinase. In the case of p38α, structural studies were carried out using the available crystal structures for p38α in both the activated (DFG-in) and inactivated (DFG-out) state. P38α does not possess a cysteine in the P-loop. The above structural studies suggested that a replacement of tyrosine with a cysteine at position 35, which is located in the P-loop, would not significantly influence the catalytic activity or stability of the kinase.

From co-pending application EP 08 01 3340, it was known that two cysteines at positions 119 and 162 of SEQ ID NO: 1 are both solvent-exposed. To avoid potential interferences of the signals recorded for two additional cysteines not located in the P-loop, these two cysteines are preferably replaced with another amino acid, preferably with an amino acid similar in size and structure, such as serine.

If a kinase homologous to p38α is used, the position of the amino acid to be replaced with cysteine may correspond to position 35 in SEQ ID NO: 1. To determine which position in a kinase corresponds to position 35 in SEQ ID NO: 1, sequence alignments of SEQ ID NO: 1 with the used kinase can be effected, e.g. using publicly available programs such as CLUSTALW.

In another preferred embodiment, the thiol- or amino-reactive fluorophore is an environmentally sensitive di-substituted naphthalene compound of which one of the two substituents is a thiol- or amino-reactive moiety. The term “environmentally sensitive” denotes the sensitivity of the fluorophore to the conditions in its environment which is expressed in an alteration in its fluorescence emission at one or more wavelengths or in its complete emission spectrum. Conditions causing such alteration are e.g. changes in the polarity or conformational changes in the activation loop and, accordingly, in the P-loop. However, changes may also occur in the P-loop without any effect on the activation loop.

The above types of fluorophores typically exhibit changes in both intensity and a shift in the emission wavelength depending on the polarity of the surrounding environment. Examples of this class of fluorophores include 6-acryloyl-2-dimethylaminonaphthalene (Acrylodan), 6-bromoacetyl-2-dimethylamino-naphthalenebadan (Badan), 2-(4′-(iodoacetamido)anilino)naphthalene-6-sulfonic acid, sodium salt (IAANS), 2-(4′-maleimidylanilino)naphthalene-6-sulfonic acid, sodium salt (MIANS), 5-((((2-iodoacetyl)amino)ethyl)amino)naphthalene-1-sulfonic acid (1,5-IAEDANS) and 5-dimethylaminonaphthalene-1-sulfonyl aziridine (dansyl aziridine) or a derivative thereof.

Other fluorophores which may be used due to their environmental sensitivity are coumarin-based compounds, benzoxadiazole-based compounds, dapoxyl-based compounds, biocytin-based compounds, fluorescein, sulfonated rhodamine-based compounds such as AlexaFluor dyes (Molecular Probes), Atto fluorophores (Atto Technology) or Lucifer Yellow. Coumarin-based fluorophores are moderately sensitive to environment and 7-diethylamino-3-(4′-maleimidylphenyl)-4-methylcoumarin (CPM) is an example. Benzoxadiazole fluorophores are also commonly used for forming protein-fluorophore conjugates and have a strong environmental dependence with 7-fluorobenz-2-oxa-1,3-diazole-4-sulfonamide (ABD-F) and N-((2-(iodoacetoxy)ethyl)-N-methyl)amino-7-nitrobenz-2-oxa-1,3-diazole ester (IANBD) as examples. PyMPO maleimide (for thiols) or succinimide ester (for amines) and various other dapoxyl dyes have good absorptivity and exceptionally high environmental sensitivity. Examples are 1-(2-maleimidylethyl)-4-(5-(4-methoxyphenyl)oxazol-2-yl)pyridinium methanesulfonate (PyMPO-maleimide), 1-(3-(succinimidyloxycarbonyl)benzyl)-4-(5-(4-methoxyphenyl)oxazol-2-yl)pyridinium bromide (PyMPO-succinimidyl ester) and Dapoxyl(2-bromoacetamidoethyl)sulphonamide. However, due to their longer, more flexible structures, these probes may effect P-loop movement or interactions between the P-loop and activation loop depending on the labelling site chosen. The applicability of the above substances depends on the individual kinase and the position of the amino acid to be labelled so that they can in principle be applied as labels as well, even if in some cases they may cause a reduced sensitivity in the methods of the invention. Matching the above substances with a suitable kinase can be performed by the skilled artisan using routine procedures in combination with the teachings of this invention.

In general, any fluorophore can be used as long as it does not inhibit the catalytic activity or interfere with the stability of the kinase. This means that the fluorophore is preferably not bulky or extended.

In a further preferred embodiment, the thiol-reactive spin-label is a nitroxide radical.

The dominant method for site-specifically labelling protein sequences with a spin-label is the reaction between methanethiosulfonate spin label and cysteine, to give the spin-labelled cysteine side chain, CYS-SL: MeS(O)2SSR+R′SH→R′SSR+MeS(O)2SH where R is the nitroxide group and R′SH is a protein with a cysteine sulfhydryl, and R′SSR is the spin-labelled protein. The cysteines for labelling are placed in the desired sequence position either through solid-phase techniques or through standard recombinant DNA techniques.

The present invention furthermore relates to a method of screening for kinase inhibitors comprising (a) providing a (fluorescently or spin-labelled or isotope-labelled) kinase according to the invention; (b) contacting said (fluorescently or spin-labelled or isotope-labelled) kinase with a candidate inhibitor; (c) recording the fluorescence emission signal at one or more wavelengths or a spectrum of said fluorescently labelled kinase of step (a) and step (b) upon excitation; or (c)′ recording the electron paramagnetic resonance (EPR) or nuclear magnetic resonance (NMR) spectra of said spin-labelled or isotope-labelled kinase of step (a) and step (b); and (d) comparing the fluorescence emission signal at one or more wavelengths or the spectra recorded in step (c) or the EPR or NMR spectra recorded in step (c)′; wherein a difference in the fluorescence intensity at at least one wavelength, preferably at the emission maximum and/or a shift in the fluorescence emission wavelength in the spectra of said fluorescently labelled kinase obtained in step (c), or an alteration in the EPR or NMR spectra of said spin-labelled or isotope-labelled kinase obtained in step (c)′ indicates that the candidate inhibitor is a kinase inhibitor.

Kinase inhibitors are substances capable of inhibiting the activity of kinases. They can more specifically inhibit the action of a single kinase, e.g. if they are allosteric inhibitors (Type III) or those binding to the allosteric site adjacent to the ATP-binding site and reaching into the ATP-binding pocket (Type II). Alternatively, an inhibitor can inhibit the action of a number of protein kinases, which is particularly the case if it binds exclusively to the ATP-binding pocket (Type I), which is very conserved among protein kinases.

A candidate inhibitor may belong to different classes of compounds such as small organic or inorganic molecules, proteins or peptides, nucleic acids such as DNA or RNA. Such compounds can be present in molecule libraries or designed from scratch.

Small molecules according to the present invention comprise molecules with a molecular weight of up to 2000 Da, preferably up to 1500 Da, more preferably up to 1000 Da and most preferably up to 500 Da.

Recording the fluorescence emission signal at one or more wavelengths or a spectrum is usually accomplished using a fluorescence spectrometer or fluorimeter. Fluorescence spectroscopy or fluorimetry or spectrofluorimetry is a type of electromagnetic spectroscopy which analyzes fluorescence, or other emitted light, from a sample. It involves using a beam of light, usually ultraviolet light, that excites the electrons in certain molecules and causes them to emit light of a lower energy upon relaxation, typically, but not necessarily, visible light.

Two general types of instruments exist which can both be employed in the method of the invention: Filter fluorimeters use filters to isolate the incident light and fluorescent light, whereas spectrofluorimeters use diffraction grating monochromators to isolate the incident light and fluorescent light. Both types utilize the following scheme: The light from an excitation source passes through a filter or monochromator and strikes the sample. A proportion of the incident light is absorbed by the sample, and some of the molecules in the sample fluoresce. The fluorescent light is emitted in all directions. Some of this fluorescent light passes through a second filter or monochromator and reaches a detector, which is usually placed at 90° to the incident light beam to minimize the risk of transmitted or reflected incident light reaching the detector. Various light sources may be used as excitation sources, including lasers, photodiodes, and lamps; xenon and mercury vapor lamps in particular. The detector can either be single-channeled or multi-channeled. The single-channeled detector can only detect the intensity of one wavelength at a time, while the multi-channeled detects the intensity at all wavelengths simultaneously, making the emission monochromator or filter unnecessary. The different types of detectors have both advantages and disadvantages. The most versatile fluorimeters with dual monochromators and a continuous excitation light source can record both an excitation spectrum and a fluorescence spectrum. When measuring fluorescence spectra, the wavelength of the excitation light is kept constant, preferably at a wavelength of high absorption, and the emission monochromator scans the spectrum. For measuring excitation spectra, the wavelength passing though the emission filter or monochromator is kept constant and the excitation monochromator is scanning. The excitation spectrum generally is identical to the absorption spectrum as the fluorescence intensity is proportional to the absorption (for reviews see Rendell, 1987; Sharma and Schulman, 1999; Gauglitz and Vo-Dinh, 2003; Lakowicz, 1999).

Nuclear magnetic resonance (NMR) is a physical phenomenon based upon the quantum mechanical magnetic properties of the nucleus of an atom. All nuclei that contain odd numbers of protons or neutrons have an intrinsic magnetic moment and angular momentum. The most commonly measured nuclei are hydrogen (¹H) (the most receptive isotope at natural abundance) and carbon (¹³C), although nuclei from isotopes of many other elements (e.g. ¹¹³Cd, ¹⁵N, ¹⁴N ¹⁹F, ³¹P, ¹⁷O, ²⁹Si, ¹⁰B, ¹¹B, ²³Na, ³⁵Cl, ¹⁹⁵Pt) can also be observed. NMR resonant frequencies for a particular substance are directly proportional to the strength of the applied magnetic field, in accordance with the equation for the Larmor precession frequency. NMR measures magnetic nuclei by aligning them with an applied constant magnetic field and perturbing this alignment using an alternating magnetic field, those fields being orthogonal. The resulting response to the perturbing magnetic field is the phenomenon that is exploited in NMR spectroscopy and magnetic resonance imaging, which use very powerful applied magnetic fields in order to achieve high spectral resolution, details of which are described by the chemical shift and the Zeeman Effect.

In the present invention, a suitable amino acid in the P-loop can be labelled with an isotope or thiol/amino-reactive small molecule containing enriched isotopes. In this case, the only signal comes from the enriched molecule on the P-loop, which is sensitive to protein conformation depending on the labelling site chosen.

Preferred isotopes are ¹³C, ¹⁵N, etc. which can be measured as 1D or 2D NMR spectra. Changes in protein conformation, e.g. due to the binding of an inhibitor will result in a shift of the NMR chemical shift(s) corresponding to the label.

Electron paramagnetic resonance (EPR) or electron spin resonance (ESR) spectroscopy, as has been briefly described above, is a technique for studying chemical species that have one or more unpaired electrons, such as organic and inorganic free radicals or inorganic complexes possessing a transition metal ion. The basic physical concepts of EPR are analogous to those of nuclear magnetic resonance (NMR), but it is electron spins that are excited instead of spins of atomic nuclei. Because most stable molecules have all their electrons paired, the EPR technique is less widely used than NMR. However, this limitation to paramagnetic species also means that the EPR technique is one of great specificity, since ordinary chemical solvents and matrices do not give rise to EPR spectra.

The EPR technique utilizes spin-labels. In this case, the kinase, to be examined is expressed in bacteria or other suitable host cells in the presence of an isotope such as ¹³C and ¹⁵N resulting in the incorporation of these isotopes throughout the entire protein as it is expressed. After purification of the isotope enriched protein, a spin label is attached to the P-loop as described above. In this case, 2D NMR spectra of the isotopes in the protein are recorded. As the P-loop and spin label change conformation, the spin label will induce a change in some of the protein signals coming from the incorporated isotopes which come into closer contact with the P-loop or spin label as inhibitors bind. Peaks would become broader as the spin label approaches.

Different EPR spectra or fluorescence emission signals at one or more wavelengths, preferably at the emission maximum, or different fluorescence emission spectra obtained in step (c) or (c)′ indicate a conformational change in the kinase caused by binding of the candidate compound. This is due to the fact that binding of a compound to the allosteric site adjacent to the ATP-binding pocket, and in some cases to the ATP-binding pocket itself, results in a perturbation of the DFG motif, a conformational change in the activation loop and, accordingly, in the P-loop, a polarity change and/or a change in the interaction of free electrons in an attached spin-label with the nuclei of adjacent atoms. Upon comparison of the EPR or NMR spectra or the fluorescence emission, the present method reveals whether a candidate compound qualifies as a suitable kinase inhibitor, e.g. not only a high-affinity inhibitor but also one which specifically inhibits the activity of one kinase. The data recorded for the kinase without a candidate inhibitor and those recorded for the kinase having been contacted with said candidate inhibitor are compared. In case of fluorescence emission signal either the signal at one or more specific wavelengths can be recorded and compared enabling for a detection of a change in the intensity of the signal at the particular wavelength(s). Alternatively, a complete spectrum can be recorded and compared enabling also for the observation of changes in the maximum emission wavelength.

Preferably, said method is effected in high-throughput format. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact with test compounds, in this case putative inhibitors, with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits inhibitory activity, said mixture of test inhibitors may be de-convoluted to identify the one or more test inhibitors in said mixture giving rise to said activity.

Alternatively, only one test inhibitor may be added to a well, wherein each test inhibitor is applied in different concentrations. For example, the test inhibitor may be tested in two, three or four wells in different concentrations. In this initial screening, the concentrations may cover a broad range, e.g. from 10 nM to 10 μM. The initial screening serves to find hits, i.e. test inhibitors exerting inhibiting activity at at least one concentration, preferably two, more preferably all concentrations applied, wherein the hit is more promising if the concentration at which an inhibitory activity can be detected is in the lower range. This alternative serves as one preferred embodiment in accordance with the invention.

Test inhibitors considered as a hit can then be further examined using an even wider range of inhibitor concentrations, e.g. 10 nM to 20 μM. The method applied for these measurements is described in the following.

The present invention furthermore relates to a method of determining the kinetics of ligand binding and/or of association or dissociation of a kinase inhibitor comprising (a) contacting a fluorescently labelled kinase according to the invention with different concentrations of an inhibitor; or (a)′ contacting a fluorescently labelled kinase according to the invention bound to an inhibitor with different concentrations of unlabelled kinase; (b) recording the fluorescence emission signal at one or more wavelengths or a spectrum of said fluorescently labelled kinase for each concentration of inhibitor and/or unlabelled kinase upon excitation; (c) determining the rate constant for each concentration from the fluorescence emission signals at one or more wavelengths or the spectra recorded in step (b) or (c1) determining the K_{d} from the fluorescence emission signal at one or more wavelengths or the spectra recorded in step (b) for each concentration of inhibitor; or (c2) determining the Kₐ or inverse K_{d} from the fluorescence emission signal at one or more wavelengths or the spectra recorded in step (b) for each concentration of unlabelled kinase; (d) directly determining the kₒₙ and/or extrapolating the k_{off} from the rate constants determined in step (c) from the signals or spectra for the different concentrations of inhibitor obtained in step (b); or (d)′ directly determining the k_{off} and/or extrapolating the kₒₙ from the rate constants determined in step (c) from the signals or spectra for the different concentrations of unlabelled kinase obtained in step (b); and optionally (e) calculating the k_{d} and/or Kₐ from kₒₙ and k_{off} obtained in step (d) or (d)′.

By contacting a labelled kinase with different concentrations of an inhibitor, and subsequently determining the fluorescence emission for each concentration applied, the binding affinity of an inhibitor can be measured. For each concentration, the ratio of bound and unbound inhibitor will be different, reflecting the increasing concentration of inhibitor but also the specific binding affinity of said inhibitor to said kinase.

The opposite approach can be followed by titrating a labelled kinase containing a bound inhibitor with unlabelled kinase with no inhibitor bound.

In chemical kinetics, a rate constant k quantifies the speed of a chemical reaction. For a chemical reaction where substance A and B are reacting to produce C, the reaction rate has the form:

$$\frac{d [ C ]}{d t} = {{k ( T ) [ A ]}^{m} [ B ]}^{n}$$

Wherein k(T) is the reaction rate constant that depends on temperature.

[A] and [B] are the concentrations of substances A and B, respectively, in moles per volume of solution assuming the reaction is taking place throughout the volume of the solution.

The exponents m and n are the orders and depend on the reaction mechanism. They can be determined experimentally.

A single-step reaction can also be described as:

$$\frac{d [ C ]}{d t} = A {{{e}^{\frac{- {E}_{a}}{RT}} [ A ]}^{m} [ B ]}^{n}$$

Eₐ is the activation energy and R is the Gas constant. Since at temperature T the molecules have energies according to a Boltzmann distribution, one can expect the proportion of collisions with energy greater than Eₐ to vary with e^{-Ea/RT}. A is the pre-exponential factor or frequency factor.

kₒₙ and k_{off} are constants that describe non-covalent equilibrium binding. When a ligand interacts with a receptor, or when a substrate interacts with an enzyme, the binding follows the law of mass action.

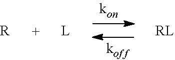

In this equation R is the concentration of free receptor, L is the concentration of free ligand, and RL is the concentration of receptor-ligand complex. In the case of enzyme kinetics, R is the enzyme, or in this case a protein kinase, and L is the substrate, or in this case a candidate or known inhibitor. The association rate constant kₒₙ is expressed in units of M⁻¹ sec⁻¹. The rate of RL formation equals R×L×kₒₙ. The dissociation rate constant k_{off} is expressed in units of sec⁻¹. The rate of RL dissociation equals RL×k_{off}. At equilibrium, the backward (dissociation) reaction equals the forward (association) reaction. Binding studies measure specific binding, which is a measure of RL. Enzyme kinetic assays assess enzyme velocity, which is proportional to RL, the concentration of enzyme-substrate complexes.

$$RL = R \cdot L \cdot \frac{{k}_{on}}{{k}_{off}}$$

The equilibrium dissociation constant, Kd is expressed in molar units and defined to equal k_{off}/kₒₙ to arrive at

$$RL = R \cdot L \cdot \frac{{k}_{on}}{{k}_{off}} = \frac{R \cdot L}{{K}_{d}}$$

The dissociation constant (K_{d}) corresponds to the concentration of ligand (L) at which the binding site on a particular protein is half occupied, i.e. the concentration of ligand, at which the concentration of protein with ligand bound (RL), equals the concentration of protein with no ligand bound (R). The smaller the dissociation constant, the more tightly bound the ligand is, or the higher the affinity between ligand and protein.

Accordingly, the association constant Kₐ, also called inverse K_{d}, is defined as 1/k_{d}. The dissociation constant for a particular ligand-protein interaction can change significantly with solution conditions (e.g. temperature, pH and salt concentration).

Depending on which sequence of steps is followed in the above method of the invention, the K_{d} or Kₐ can be measured directly or indirectly.

For directly measuring the K_{d} or the Kₐ, respectively, step (c1) or (c2) which is the last step for this type of measurement follows step (b). This type of measurement is called endpoint measurement and also illustrated in the appended examples. Unlike for indirectly determining K_{d} or Kₐ through calculation using rate constants, the final fluorescence emission at equilibrium is measured rather than the fluorescence change over time. These measurements can be used to generate a binding curve using different inhibitor concentrations (for determining K_{d}) or concentrations of unlabelled kinase (for determining Kₐ). From these curves, K_{d} or Kₐ can be obtained directly.

For indirectly obtaining K_{d} or Kₐ, the rate constants from the fluorescence emission signal at one or more wavelengths or the spectra recorded in step (b) have to be determined for each concentration as done in step (c). Depending the type of titration, i.e. titration of labelled kinase with inhibitor or titration of labelled kinase bound to inhibitor with unlabelled kinase, either kₒₙ or k_{off} can be determined directly from the measured rate constants. For determining kₒₙ, step (d) is applied which also enables for extrapolation of k_{off}. Accordingly, step (d)′ is applied for directly determining k_{off} which in turn enables for extrapolation of kₒₙ. From kₒₙ and/or k_{off} obtained in steps (d) or (d)′, the K_{d} and/or Kₐ can be calculated according to the equations discussed above.

The above method may also be applied in high-throughput screens. If a compound exerting inhibitory activity on a kinase has been identified, e.g. using the method of screening for kinase inhibitors of the invention, the present method can be used to further characterize said inhibitor. For example, the high-throughput format can be used to determine the Kₐ or k_{d} from the fluorescence emission signal at one or more wavelengths for multiple different concentrations of inhibitors (variant (a)) or, unlabelled kinase (variant (b)). Concentration ranges to be tested reach for example from 10 nM to 20 μM such that repeating series of 1, 2 and 5 (i.e. 10, 20, 50, 100, 200, 500 nM, etc.) between the concentrations assessed.

In a different embodiment, the present invention relates to a method of determining the dissociation or association of a kinase inhibitor comprising (a) contacting a spin-labelled or isotope-labelled kinase according to the invention with different concentrations of an inhibitor; or (a)′ contacting a spin-labelled or isotope-labelled kinase according to the invention bound to an inhibitor with different concentrations of unlabelled kinase; (b) recording the EPR or NMR spectrum of said spin-labelled or isotope-labelled kinase for each concentration of inhibitor and/or unlabelled kinase; and (c) determining the K_{d} from the EPR or NMR spectra recorded in step (b) for the different concentrations of inhibitor; or (c)′ determining the Kₐ from the EPR or NMR spectra recorded in step (b) for the different concentrations of unlabelled kinase.

Similar to the method disclosed further above relating to determining the kinetic constants using fluorescently labelled kinase, the present method allows for the direct determination of the association or dissociation constants for the reaction a kinase and an inhibitor. Unlike for fluorescently labelled kinases, the instrumental limitations and time required to collect NMR and EPR measurements are, in most cases, not compatible with the fast time scale of inhibitor binding and do not allow the direct determination of kₒₙ or k_{off}. Determinations for compounds which require several hours to bind to the kinase may also be possible.

The methods of the invention relating to determining kinetic data can also be applied to a high-throughput format. For example, a potential inhibitor identified with the screening method of the invention described above can be further characterized in that different concentrations of said inhibitor are applied to the kinase to determine the Kd. Suitable but not limiting concentration ranges for the inhibitor are between 10 nM and 20 μM.

More focused concentration ranges applied in the high-throughput format may serve to obtain more sensitive Kd measurements, e.g. with the cuvette approach and real-time kinetics measurements as done in the appended examples, by determining kₒₙ and k_{off}.

The present invention furthermore relates to a method of generating mutated kinases suitable for the screening of kinase inhibitors comprising (a) replacing solvent exposed amino acids having a free thiol or amino group, if any, present in a kinase of interest outside the P-loop or amino acids having a free thiol or amino group at an unsuitable position within the P-loop with an amino acid not having a free thiol or amino group; (b) mutating an amino acid in the P-loop of said kinase of interest to an amino acid having a free thiol or amino group if no amino acid having a free thiol or amino group is present in the P-loop; (c) labelling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity and/or does not interfere with the stability of the kinase; (d) contacting the kinase obtained in step (c) with a known inhibitor of said kinase; and (e) recording the fluorescence emission signal at one or more wavelengths or a spectrum of said fluorescently labelled kinase of step (c) and (d) upon excitation or (e)′ recording the EPR or NMR spectra of said spin-labelled kinase of step (c) and (d); and (f) comparing the fluorescence emission signal at one or more wavelengths or the spectrum recorded in step (e) or the EPR or NMR spectra recorded in step (e)′; wherein a difference in the fluorescence intensity at at least one wavelength, preferably the emission maximum and/or a shift in the fluorescence emission wavelength in the spectra of said fluorescently labelled kinase obtained in step (e), or an alteration in the EPR or NMR spectra of said spin-labelled or isotope-labelled kinase obtained in step (e)′ indicates that the kinase is suitable for the screening for kinase inhibitors.

Adapted to a high-throughput format, multiple kinases or differently labelled variations of the same kinase can be screened.

The term “unsuitable position” in accordance with the present invention denotes a position in the P-loop which was shown to be not suitable for an amino acid labelled according to the invention. This can be due to a decreased sensitivity of the label to changes in its environment or due to predictions based on structural considerations that said position would result in a kinase with a label with decreased sensitivity. The term also encompasses amino acids positioned at a potentially suitable position, wherein a different position is deemed more appropriate. As soon as the number of amino acids having a free thiol or amino group in the P-loop exceeds one, amino acids deemed as unsuitable should be mutated.

Mutating an amino acid includes replacing or deleting said amino acid with another amino acid, provided that said mutation does not result in an inhibited catalytic activity or an interference with the stability of the resulting kinase. Step (b) is carried out if no amino acid having a free thiol or amino group is present in the P-loop of said kinase of interest. The amino acid which is inserted or which replaces another amino acid has to have a free thiol or amino group in order to be labelled.

In a preferred embodiment of the methods of the present invention, the kinase inhibitor binds either exclusively to the allosteric site adjacent to the ATP binding site of the kinase or extends from the allosteric site into the ATP site. These types of inhibitors are also called Type III or Type II inhibitors, respectively. They bind to kinases with higher specificity as compared to Type I inhibitors which bind to the ATP-pocket of the kinase, which is highly conserved in structure among all kinases.

As demonstrated in the examples, the present invention provides means to differentiate between ATP-competitive and non-ATP-competitive inhibitors, enabling for a rapid election of specific inhibitors. The invention is designed to detect the movement of the P-loop of the kinase, which is caused by movement of the activation loop and is therefore sensitive to all Type II and Type III inhibitors. Furthermore, certain Type I inhibitors exerting a specific binding mode (see above) or directly interact with the P-loop with the kinase in the DFG-in conformation are detected. Only measurement of the fluorescence change over time (i.e. not an endpoint measurement) resulting in the determination of the rate of binding can allow Type I inhibitors to be distinguished from Type II and Type III inhibitors. As presented in one of the examples below, detected ATP-competitive inhibitors produce an instantaneous fluorescence change (typically <5 sec) while Type II and Type III inhibitors bind much slower (seconds to several minutes).

In another preferred embodiment of the kinase or the methods of the present invention, the kinase is labelled at a cysteine naturally present or introduced into the P-loop.

The abundance of cysteines in proteins is usually very low, so that a kinase of the invention can be prepared in a straightforward manner by replacing an amino acid in the P-loop with cysteine and optionally replacing solvent-exposed cysteines with other amino acids. Amino acids containing reactive amines, such as histidine, arginine or lysine or derivatives thereof, are much more abundant and are readily found at the protein surface where they are in contact with the surrounding solvent. Thus, it is preferable to use thiol-reactive labels which can specifically react with an introduced cysteine.

In a more preferred embodiment, the method of screening for kinase inhibitors or the method of generating mutated kinases further comprises step (c1) measuring a fluorescence intensity ratio of two wavelengths recorded in step (c) and obtaining the ratio of the normalized intensity change to the average intensity change (ΔI_{std}). Additionally or alternatively, the maximum standard intensity change (ΔRₘₐₓ) between a kinase labelled according to the invention with inhibitor bound and one without inhibitor may be assessed. A candidate compound is considered a kinase inhibitor or the fluorescent-labelled kinase is considered suitable for the screening for kinase inhibitors if (ΔI_{std}) is >0.25, and/or (ΔRₘₐₓ) is >0.75 and the Z-factor is >0.5. This embodiment relates to the extension of the methods of the present invention to high-throughput scale as described above.

ΔI_{std} is the ratio of normalized intensity change to average intensity of the fluorescence emission. According to de Lorimier et al. (2002), ΔI_{std} is one of the most important criteria for characterizing a fluorescent protein conjugate as suitable for sensitive fluorescence spectroscopy. Ideally, the ΔI_{std} should have a value >0.25 and is calculated by:

$$Δ {I}_{std} = \vert \frac{2 ( {I}_{1} ( {λ}_{std} ) - {I}_{2} ( {λ}_{std} ) )}{{I}_{1} ( {λ}_{std} ) + {I}_{2} ( {λ}_{std} )} \vert$$

where λstd=(λmax, unbound+λmax, saturated)/2 and I1, I2 are the fluorescence intensities at λstd of each spectrum respectively.

ΔRₘₐₓ is the maximum standard intensity change of the fluorescence emission between saturated and unsaturated kinase. According to (de Lorimier et al., 2002), ΔRₘₐₓ is another important criteria for characterizing a fluorescent protein conjugate as suitable for sensitive fluorescence spectroscopy. Ideally, the ΔRₘₐₓ should have a value >1.25 and is calculated by:

$$Δ R = \vert \frac{{}_{}^{o}{A}{}_{1}^{}}{{}_{}^{o}{A}{}_{2}^{}} - \frac{{}_{}^{∞}{A}{}_{1}^{}}{{}_{}^{∞}{A}{}_{2}^{}} \vert$$

where ∘A1, ∘A2 are the areas in the absence of ligand, and ∞A1, ∞A2 are the areas in the presence of saturating ligand. A computer program can be used to enumerate ΔR for all possible pairs of wavelength bands in the two spectra, to identify the optimal sensing condition, defined as the maximum value of ΔR.

The Z-factor is a statistical measure of the quality or power of a high-throughput screening (HTS) assay. In an HTS campaign, large numbers of single measurements of unknown samples are compared to well established positive and negative control samples to determine which, if any, of the single measurements are significantly different from the negative control. Prior to starting a large screening campaign, much work is done to assess the quality of an assay on a smaller scale, and predict if the assay would be useful in a high-throughput setting. The Z-factor predicts if useful data could be expected if the assay were scaled up to millions of samples. The Z-factor is calculated by:

$$Zf actor = 1 - \frac{3 \times ( {σ}_{p} + {σ}_{n} )}{\vert {μ}_{p} - {μ}_{n} \vert}$$

wherein both the mean (μ) and standard deviation (σ) of both the positive (p) and negative (n) controls (μₚ,σₚ,μₙ,σₙ, respectively) are taken into account.

The measurement of ΔI_{std} and ΔRₘₐₓ as well as the determination of the Z-factor may prove useful in determining whether the label chosen is suitable in the screening for inhibitors. De Lorimier discusses that the measured kinetics and K_{d} obtained with a fluorescent tagged protein will depend on the protein, the ligand and the fluorophore used. Therefore, the same inhibitor binding to the same kinase could give different k_{d} values depending on the label used. The determination of the above values might indicate whether the label chosen is appropriate or whether a different label should be used.

In a further preferred embodiment, the fluorophore or spin-label is not located at or adjacent to phosphorylation sites known or predicted to exist in the labelled kinase. This ensures that the labelling does not interfere with the dynamics of the P-loop or the normal activity and regulation of the kinase which is largely affected by phosphorylation and dephosphorylation.

In another preferred embodiment, said candidate amino acid in the P-loop is identified based on structural and/or sequence data available for said kinase.

For some kinases, structural data, e.g. in the form of crystal or NMR structures is available, wherein the kinase is captured in the activated and/or inactivated state. If such data is available for a kinase, this facilitates the choice of the amino acid position in the P-loop to be replaced for labelling purposes. The actual choice is based on which residue tends to exhibit the most movement in the P-loop in response to ligand binding and/or conformational changes in the position preceding the third Gly of the G-X-G-X-X-G motif, which is most often a Tyr or Phe. If this residue is a Tyr and is known to be phosphorylated in a particular kinase, which is rarely the case, labelling of this position will likely disrupt kinase activity and makes this position unsuitable in the present invention. Similarly, contacts of the amino acid in said position with other amino acids are also examined. If said contacts are deemed essential for the catalytic activity or stability of the kinase, the position is in most cases not suitable for replacement. Additionally, the choice is based on the distance which a particular amino acid will move as the protein changes conformation such that greater distances increase the chance that an environmental change will be detected. However, although distance moved is an indicator of whether a particular position may be useful for labelling, it is the actual change in environment which will correlate directly with the observed changes detected by the attached label.

In a preferred embodiment, the methods of the present invention relating to screening for inhibitors, determining kinetic parameters such as association and dissociation and generating a mutated kinase are combined to obtain a straightforward methodology to obtain specific inhibitors for different kinases. In this regard, any preferred embodiment of a method of the invention may be combined with embodiments of other methods of the invention. In a more preferred embodiment of this aspect, an initial screen is carried out using the method of high-throughput screening for kinase inhibitors, followed by a screen using a wide range of concentrations of inhibitors as described above with the method of the invention for determining the kinetics of ligand binding and/or association or dissociation. The latter step is carried out, inter alia, to get an indication of the K_{d} and/or Kₐ value. This step is again repeated by carrying out measurements with a more focused concentration range for more precise measurements of the K_{d} or Kₐ. These measurements may be carried out either as a titration series with the cuvette approach and/or real-time kinetic measurements in cuvettes (kₒₙ and k_{off}) to further characterize each inhibitor. Alternatively or additionally, the binding mode of a compound identified as a kinase inhibitor may be further characterized by crystallizing it in complex with the kinase of interest, which provides the clearest details. Optionally, this sequence of methods is transferred to other kinases or the same kinase labelled differently. This embodiment is designed to enable for high-throughput screening to screen for and characterize a high number of inhibitors in multiple kinases or differently labelled variations of the same kinase.

More specifically, such a combined method is a method for identifying a kinase inhibitor binding either partially or fully to the allosteric site adjacent to the ATP binding site of a kinase comprising (a) screening for an inhibitor according to the method of screening for kinase inhibitors of the invention, and (b) determining the rate constant of an inhibitor identified in step (a) to a kinase, wherein a rate of binding of <0.140 s⁻¹ determined in step (b) indicates that the kinase inhibitor identified binds either partially or fully to the allosteric site adjacent to the ATP binding site of the kinase. Rate constants of >0.140 s⁻¹ indicate that the kinase inhibitor identified binds in the ATP binding site and does not extend into the adjacent allosteric site. The rate constant is correlated to the reaction time (rate of binding) t_{1/2}: t_{1/2}=ln(2)/k_{obs}. Accordingly, a rate constant (k_{obs}) of <0.140 s⁻¹ corresponds to a reaction time t_{1/2} of >5 s.

The rate constant or rate of binding is preferably determined using the properties of the labelled kinase of the invention. For example, the kinase of the invention can be contacted with an inhibitor and, depending on the label, the fluorescence emission signal of a fluorescently labelled kinase at one or more wavelengths or the electron paramagnetic resonance or nuclear magnetic resonance spectra of a spin-labelled or isotope-labelled kinase can be recorded over time. This corresponds to steps (a) to (c) of the method of determining the kinetics of ligand binding and/or of association or dissociation of a kinase inhibitor of the invention or steps (a) and (b) of the method of determining the dissociation or association of a kinase inhibitor of the invention. In case the rate of binding, i.e. the measurable changes in fluorescence or in the NMR or EPR spectra, is more than 5 seconds after application of the inhibitor, this indicates that the inhibitor is a type II or type III inhibitor.

In another preferred embodiment of the method for screening of kinase inhibitors, the method further comprises (subsequently) optimizing the pharmacological properties of a candidate compound identified as inhibitor of said kinase.

Methods for the optimization of the pharmacological properties of compounds identified in screens, generally referred to as lead compounds, are known in the art and comprise a method of modifying a compound identified as a lead compound to achieve: (a) modified site of action, spectrum of activity, organ specificity, and/or (b) improved potency, and/or (c) decreased toxicity (improved therapeutic index), and/or (d) decreased side effects, and/or (e) modified onset of therapeutic action, duration of effect, and/or (f) modified pharmacokinetic parameters (absorption, distribution, metabolism and excretion), and/or (g) modified physico-chemical parameters (solubility, hygroscopicity, color, taste, odor, stability, state), and/or (h) improved general specificity, organ/tissue specificity, and/or (i) optimized application form and route by a. esterification of carboxyl groups, or b. esterification of hydroxyl groups with carboxylic acids, or c. esterification of hydroxyl groups to, e.g. phosphates, pyrophosphates or sulfates or hemi-succinates, or d. formation of pharmaceutically acceptable salts, or e. formation of pharmaceutically acceptable complexes, or f. synthesis of pharmacologically active polymers, or g. introduction of hydrophilic moieties, or h. introduction/exchange of substituents on aromates or side chains, change of substituent pattern, or i. modification by introduction of isosteric or bioisosteric moieties, or j. synthesis of homologous compounds, or k. introduction of branched side chains, or l. conversion of alkyl substituents to cyclic analogues, or m. derivatization of hydroxyl group to ketales, acetales, or n. N-acetylation to amides, phenylcarbamates, or o. synthesis of Mannich bases, imines, or p. transformation of ketones or aldehydes to Schiff's bases, oximes, acetales, ketales, enolesters, oxazolidines, thiazolidines or combinations thereof. Prior to modifying the candidate compound, several analysis/predictive tools such as (i) docking of proposed molecules into already known crystal structures, (ii) docking of proposed molecules into homology models (modeled structures generated based on the known crystal structure of the closest homologue of the target protein) and/or (iii) crystallization of the kinase-inhibitor complex may be applied in order to characterize the exact binding mode of the candidate compound to the kinase of interest and to predict the effect of certain chemical modifications to the candidate compound on its inhibitory properties against the kinase.

The modifications effected to the candidate compound may then again be further analyzed by any of the techniques listed above, so that after a number of such cycles, the properties of the candidate compound have been optimized.

The various steps recited above are generally known in the art. They further include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLE 1

### Selection of a Suitable Kinase

Applicants chose to work with p38α to develop this assay for the following reasons: i) the abundance available of structural information, ii) the availability of crystal structures in both its active and inactive conformations (FIG. 1A.) and iii) the availability of tight binding Type II & III allosteric inhibitors. In the first step, the crystal structures of p38α were closely examined to identify suitable fluorophore attachment sites that would detect allosteric binders. Candidate residues for this mutation must be solvent exposed to enable the attachment of a fluorophore by Michael addition, and exhibit significant movement upon ligand binding. Care was also taken to not choose residues that are critical to maintaining protein stability, catalytic activity or residues in the vicinity of known phosphorylation sites.

A position in P-loop (Tyr35) was selected and subsequently mutated into a cysteine residue (FIG. 1B.,C.). Acrylodan was selected as the fluorophore due to its relatively small size (comparable to a tryptophan side chain), its high sensitivity to polarity changes, its commercial availability and relatively low price. Acrylodan is also known to produce a robust response and should detect movements of the activation loop upon binding of allosteric inhibitors (FIG. 1D.). Before labelling the protein, it was necessary to reduce the chances of fluorophore attachment to any other solvent exposed cysteine residues. Again, structural information was used to locate 4 reduced cysteine residues in p38α. Two of these cysteine are buried within the protein while the other two were solvent-exposed and conservatively mutated into serine. Lastly, a F327L mutation was incorporated to partially activate (Askari et al., 2007: Avitzour et al., 2007) the acrylodan-labelled p38α (ac-p38α) for use in enzyme activity assays, if desired, but is not necessary for functionality of the assay itself.

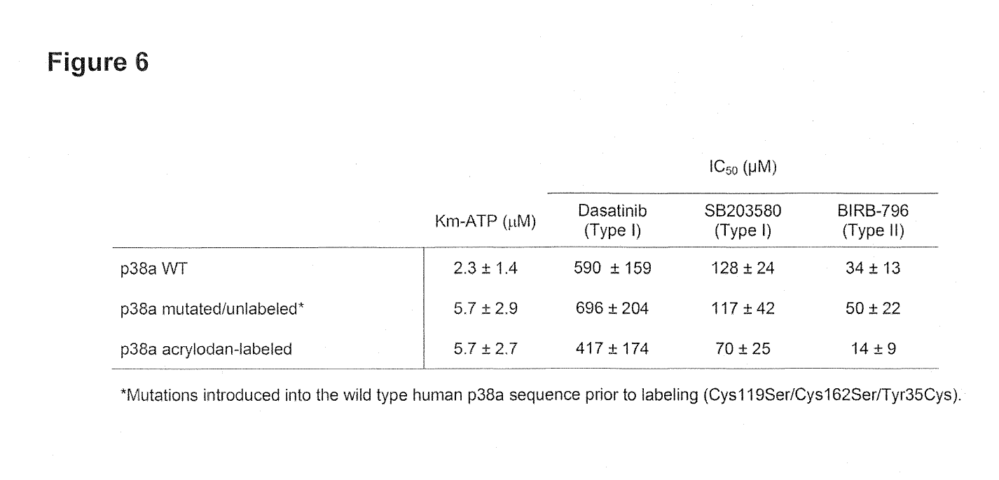

Replacing a tyrosine with the comparably sized acrylodan-labelled cysteine was shown to be well tolerated by the kinase by using an activity-based assay to measure and compare the ATP-Kₘ of each phosphorylated active p38α variant (mutated/unlabelled and acrylodan-labelled) to that of wild-type p38α (FIG. 6). Likewise, no significant changes in the IC50 values of three known p38α inhibitors upon mutation or labelling of the glycine-rich loop were observed, demonstrating that the present invention ultimately provides similar affinity data for detected ligands as the assay described in EP 08 01 3340 and EP 08 02 0341.

### EXAMPLE 2

### Protein Labelling and Fluorescence Characterization

### Protein Labelling

A p38α construct containing 4 total mutations (2 cysteine→serine, and the introduction of a cysteine for labelling) was transformed into the BL21(DE3) E. coli strain, overexpressed, purified by affinity, anion exchange and size exclusion chromatography and the pure protein was subsequently used for labelling. Protein and free acrylodan were combined at a 1:1.5 ratio and allowed to react in the dark overnight at 4° C. The conjugated protein (ac-p38α) was concentrated, aliquoted and frozen at −20° C. Mono-labelling of 100% of the protein was verified by ESI-MS. Confirmation of the correctly labelled cysteine is currently being performed by analyzing the tryptic fragments of unlabelled and labelled p38α following a combination of HPLC and ESI-MS or MALDI.

### Fluorescence Characterization

Following labelling, the fluorescent properties of the probe were characterized and initial experiments were carried out using various derivatives of the pyrazolourea Type II allosteric inhibitor, BIRB-796 (Pargellis et al., 2002; Dumas et al., 2000 (a and b); Moss et al., 2007; Regan et al., 2002; Regan et al., 2003). The ac-p38α protein labelled on the P-loop shows a modest red-shift from 475 nm to 512 nm with ligand binding (FIG. 2A). Measuring a ratio of two wavelengths (R=512 nm/475 nm) allows the possibility of eliminating dilution errors between different samples (FIG. 2B). Using these two wavelengths, average Z-factors of 0.53±0.04 can be calculated. Similarly, a small maxima present at 445 nm is relatively insensitive to ligand binding, and as a result, the ratio of two wavelength (R=445 nm/475 nm) can also be used for this purpose. Using these two wavelengths, average Z-factors of 0.85±0.05 can be calculated. A large change at 475 nm also allows for the possibility of making single-wavelength kinetic measurements (FIG. 2C-D).

Using the wavelengths 512 nm and 475 nm, the normalized intensity change compared to average intensity (ΔI_{std}) was determined to range between 0.55-1.57 for all detected inhibitor types and the maximum standard intensity change (ΔRₘₐₓ) between saturated and unsaturated ac-p38α ranged between 0.23-0.56 for all detected inhibitor types. These are two of the most important criteria for fluorescence spectroscopy (de Lorimier et al., 2002). The ΔI_{std} values together with the determined Z factors for the two different ratiometric readouts characterize this as a suitable probe for use in fluorescence assays.

### EXAMPLE 3

### Endpoint and Kinetic Measurements—Methods

To characterize the present labelling strategy, p38α labelled with acrylodan at the glycine-rich loop (50 nM) was screened against a small subset (˜400) of compounds based on scaffolds that are generally known to be privileged for binding to the DFG-in or DFG-out conformation of kinases. The kinase was pre-incubated with various concentrations of inhibitor before endpoint fluorescence measurements were carried out in either polystyrene cuvettes or 384-well plates to determine the K_{d} of each compound. A standard buffer (50 mM Hepes, 200 mM NaCl, pH 7.45) was used for all experiments. For cuvette measurements, incubations were carried out overnight in the dark at 4° C. for p38α. For HTS formats, incubations were carried out for up to 5 h at room temperature. Long incubation times are needed to account for the time-dependence of Type II inhibitor binding to p38α (Pargellis et al., 2002).

In the cuvette format, a series of cuvettes containing different amounts of inhibitor were prepared using inhibitor stocks (0.01, 0.1, 1.0, and 10.0 mM in DMSO). All measurements of the cuvettes were made with a JASCO FP-6500 fluorescence spectrophotometer (JASCO GmbH, Gross-Umstadt, Germany). A Tecan Safire^{II} (Tecan Deutschland GmbH, Germany) was used to measure the fluorescence read-out in the 384-well plate format. The % v/v DMSO did not exceed 0.2% in cuvettes and was 5% v/v in 384-well plates. In the case of p38α, average Z′ factors of 0.67±0.05 (n=6) and 0.64±0.10 (n=6) were determined for the cuvette and 384-well formats, respectively, using saturating amounts of BIRB-796 or sorafenib as a positive control for a ligand that induces glycine-rich loop movement. Vehicle (DMSO) was used as the negative control.

For acrylodan-labelled p38α, ratiometric fluorescence values (R=I_{λ512}/I_{λ475}) enabled reliable binding curves of detected compounds to be plotted, allowing for direct determination of the K_{d} of ligand binding. It should be noted that binding modes of detected molecules can initially be assessed in HTS formats by measuring plates from primary and/or secondary screens at different time points. Compounds that change the maximum ratiometric signal or K_{d} over time are likely to be Type II/III inhibitors. Alternatively, kinetics of the association (kₒₙ) and dissociation (k_{off}) of selected compounds can be determined using cuvettes. To determine kₒₙ, a mini stir bar was placed in the bottom of each cuvette to ensure rapid mixing as inhibitor was delivered through the injection port located above the cuvette. Fluorescence changes were monitored at 475 nm for p38α in real-time using a JASCO FP-6500 fluorescence spectrophotometer (JASCO GmbH, Groβ-Umstadt, Germany). Nearly instantaneous binding kinetics (<5 s) are characteristic of Type I inhibitors, while slower kinetics (>10 s) indicate the slower binding of Type II or Type III inhibitors to the DFG-out conformation. Following binding, k_{off} was determined by adding a 10-fold excess of unlabelled kinase to shift the binding equilibrium away from the labelled kinase. Addition of excess unlabelled kinase causes the inhibitor to redistribute and dissociate from acrylodan-labelled p38α, resulting in a recovery of the fluorescence signal. All binding and dissociation curves were fit to a single exponential equation: F(t)=F(∞)+F(0) exp(−t*k_{obs}), where t is time, F(0) is the initial fluorescence intensity, and F(∞) is the fluorescence at t=∞. The half-time of fluorescence decay (t_{1/2}) was calculated with the following equation: t_{1/2}=ln 2/k_{obs}.

### EXAMPLE 4

### Kinase Expression & Purification

The p38α construct was cloned into a pOPINE, pOPINF or pOPINM vector and was transformed as an N-terminal His-tag construct with Precision Protease cleavage site into BL21(DE3) E. coli, BL21(DE3)Codon+RIL E. coli or BL21(DE3)Rosetta E. coli. Cultures were grown at 37° C. until an OD600 of 0.6, cooled in 30 min to RT and then induced with 1 mM IPTG for overnight (˜20 hrs) expression at 18° C. while shaking at 160 rpm. Cells were lysed in Buffer A (50 mM Tris pH 8.0, 500 mM NaCl+5% glycerol+25 mM imidazole) and loaded onto a 30 mL Ni-column (self-packed), washed with 3 CV of Ni Buffer A and then eluted with a 0-50% linear gradient using Ni Buffer B (Ni Buffer A+500 mM imidazole) over 2 CV. The protein was cleaved by incubating with PreScission Protease (50 μg/mL final concentration) in a 12-30 mL capacity 10-MWCO dialysis cassette (Thermo Scientific) overnight at 4° C. in Dialysis Buffer (50 mM Tris pH 7.5, 5% glycerol, 150 mM NaCl, 1 mM EDTA, 1 mM DTT). The protein was then centrifuged for 15 min at ˜13,000 rpm to remove any precipitate that may have formed during the cleavage step. The supernatant was then taken and diluted at least 4-fold in Anion Buffer A (50 mM Tris pH 7.4, 5% glycerol, 50 mM NaCl, 1 mM DTT) and loaded onto a 1 mL Sepharose Q FF column (GE Healthcare) and washed with 10 CV of Anion Buffer A. The protein was eluted with a 0-100% linear gradient of Anion Buffer B (Anion Buffer A+600 mM NaCl) over 20 CV. The protein was pooled and concentrated down to 2 mL and passed through a Sephadex HiLoad 26/60 Superdex 75 column equilibrated with Size Exclusion Buffer (20 mM Tris pH 7.4, 5% glycerol, 200 mM NaCl, 1 mM DTT) at a rate of 2 mL/min. The eluted protein was then concentrated to ˜10 mg/mL, aliquoted and frozen at −80° C.

### EXAMPLE 5

### Real-Time and Endpoint Fluorescence Measurements Using ac-p38α Labelled on the Glycine-Rich Loop Enables Detection of Type II/III Inhibitors and some Type I Inhibitors

To compare the present invention with the method described in EP 08 01 3340 and EP 08 02 0341 in which the activation loop of p38α was labelled, and to demonstrate that labelling the glycine-rich loop with acrylodan serves as a reliable alternative approach, the Type II p38α inhibitor BIRB-796 was used to characterize the fluorescence response. Endpoint measurements were carried out by measuring the emission spectrum of glycine-rich loop labelled p38α in the presence of increasing concentrations of BIRB-796 (FIG. 2A). Subsequently, a ratiometric fluorescence value (R=I_{λ512}/I_{λ475}) was plotted on a logarithmic scale against the concentration of ligand to directly determine K_{d}=9.5±2.7 nM for BIRB-796 (FIG. 2B), which is similar to the value of 7.5±2.3 nM obtained with p38α labelled at the activation loop and demonstrates the reliability of K_{d} values for DFG-out binders obtained with this alternative approach.

A significant change of emission at 475 nm also made it possible to study the kinetics of dissociation and association in real-time for different concentrations of ligand. Upon binding of both Type II (e. g. BIRB-796) and Type III (e. g. RL36) inhibitors, acrylodan emission at 475 nm decreases resulting in a red-shift of the maximum emission wavelength in the bound state (FIGS. 2A and 3A-left panel). At equilibrium, the emission spectra of P-loop labelled p38α changes such that the emission intensities at 512 nm and 475 nm are nearly equal (i.e. R=512/475 nm usually has a value of ˜1.0). Using this ratiometric fluorescence output, endpoint equilibrium measurements can be made to directly obtain the K_{d} of these ligands (FIGS. 2B and 3A-right) by plotting the fluorescence data against a logarithmic scale of inhibitor concentration. This response is characteristic of any Type II or III inhibitor which occupies the allosteric site adjacent to the ATP-site and requires a conformational change in the activation loop. As described above, this conformational change induces a characteristic change in the P-loop which alters the fluorescence of acrylodan in a specific manner. Type I ligands known to stabilize the DFG-out conformation by stacking between the P-loop and the Phe of the DFG motif are also sensitively detected and induce even larger intensity losses at 475 nm when compared to Type II/III ligands (FIG. 3B-left).

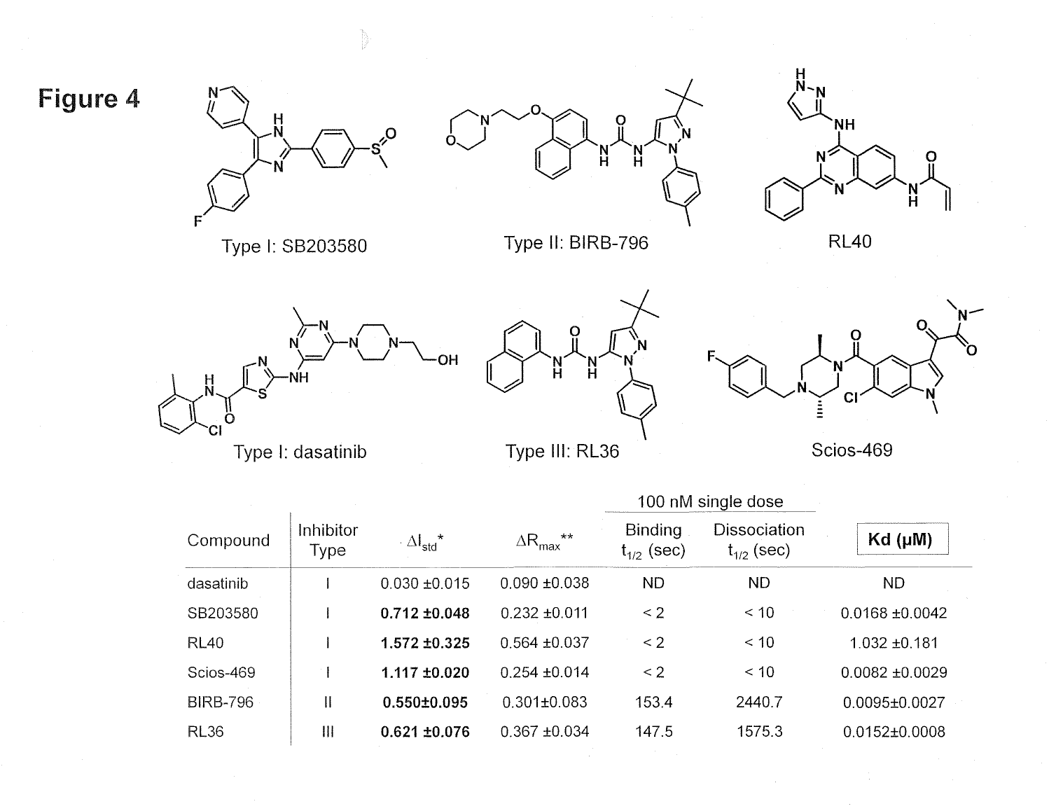

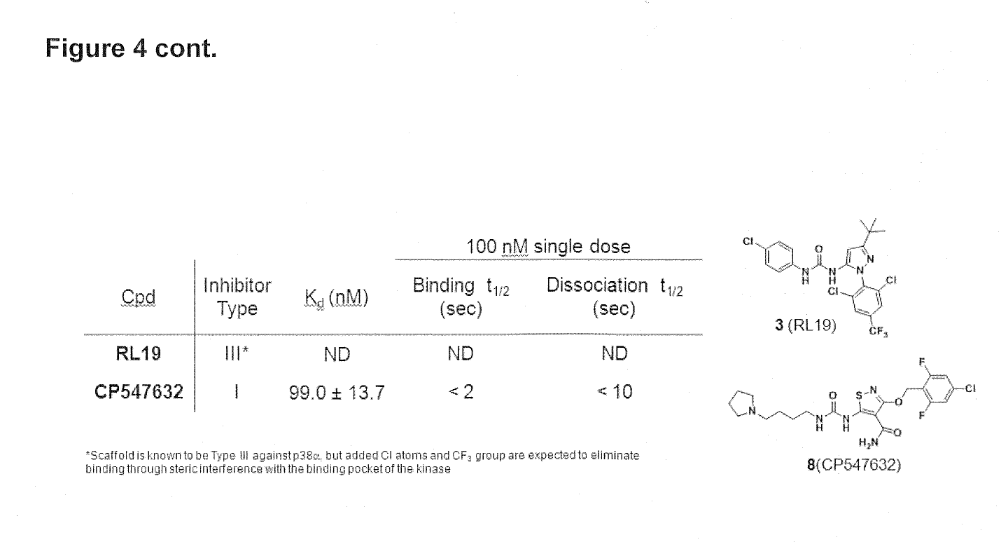

The discrimination of such Type I ligands from Type II or III ligands is easily accomplished by examining the binding kinetics of each ligand. Kinetic measurements are made by monitoring the decrease in emission intensity at a single wavelength (475 nm) upon addition of ligand to a suspension of the labelled kinase in buffer. In the case of Type II inhibitors such as BIRB-796 (FIG. 2C) and Type III inhibitors such as RL36 (FIG. 3a-middle), the kinetics of binding is significantly slower than that of Type I inhibitors, regardless of whether the Type I inhibitor stabilizes the DFG-in or DFG-out conformation (see kinetic values in FIG. 4). The slower fluorescence decays resulting from the addition of different Type II or III inhibitors can be easily fit to a first-order decay function to obtain k_{obs} (FIG. 2C and FIG. 3a-middle). Experimentally determined k_{obs} values can then be plotted to determine kₒₙ for BIRB-796 any ligand as described in EP 08 01 3340 and EP 08 02 0341. The kₒₙ determined for BIRB-796 to be ˜4.0×10⁴ M⁻¹ s⁻¹. The fluorescence decays of Type I inhibitor binding are too fast to fit accurately without the use of stopped-flow fluorescence spectroscopy. Extraction of inhibitors from ac-p38α using an excess of unlabelled p38α results in an upward change in the fluorescence intensity at 475 nm which was also fit to a first-order function fluorescence to allow direct determination of k_{off} (FIG. 2D). These measurements demonstrate the reversibility of the fluorescence response and demonstrate the changing equilibrium which exists between the DFG-in and DFG-out conformations. Regardless of inhibitor type, the rate of dissociation of the ligand from the protein is always slower than the rate of binding, a well-known observation, particularly for p38α (Pargellis et al. 2003).

After the fluorescence response of glycine-rich loop-labelled p38α was characterized, the assay was adapted to HTS formats aimed at the sensitive detection of Type II and Type III inhibitors in a small subset of compounds available (Kluter et al., 2009; Getlik et al., 2009; Michalczyk et al., 2008; Pawar et al., 2010; Sos et al., 2010) comprising various scaffolds known to be privileged for binding to the DFG-in or DFG-out conformation of kinases. After an initial pre-screen at three fixed inhibitor concentrations (0.5, 5, and 50 μM), selected compounds, some of which were derivatives of known p38α inhibitors, were subjected to further studies using concentration-dependent direct binding measurements to determine kₒₙ, k_{off}, and K_{d} (FIGS. 3 and 4) and to further compare affinities of compounds detected with the method of the invention and the method disclosed in EP 08 01 3340 and EP 08 02 0341 as a means of validation.

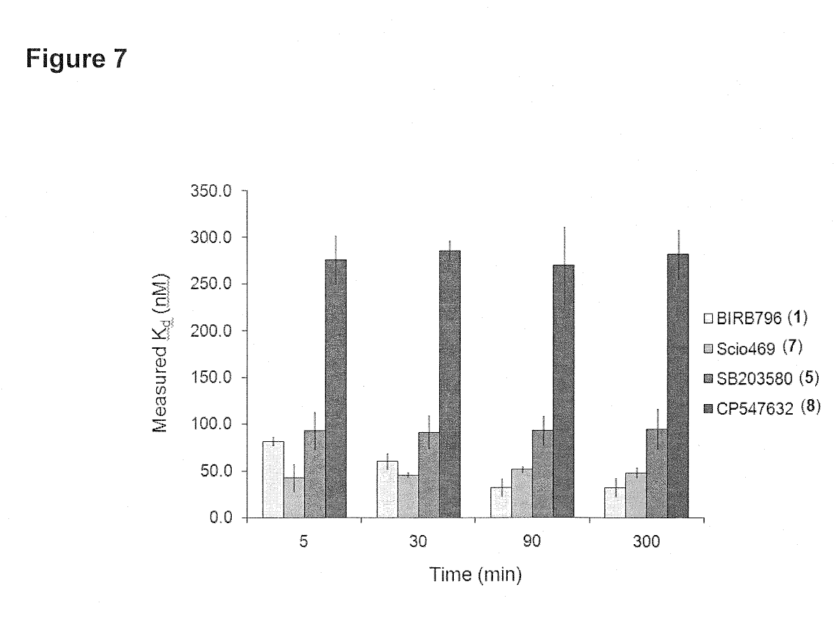

As expected, the known DFG-out binder BIRB-796 showed a distinct time dependence (FIG. 7) over a period of 5 h and was found to have a K_{d} value similar to that obtained using activation loop-labelled p38α in EP 08 02 0341. Additionally, BIRB-796 and RL36 were found to differ mainly with respect to k_{off} rather than kₒₙ as the primary determinant for affinity, which is a well-characterized observation for the binding of pyrazolourea-based compounds to the DFG-out conformation of p38α (Pargellis et al., 2002; Simard et al., 2009). The known Type I p38α inhibitor SB203580 was also sensitively detected and had a K_{d} value comparable to that disclosed in EP 08 02 0341. EP 08 02 0341 discloses that, despite adopting a Type I binding mode and contacting the kinase hinge region, SB203580 binds to p38α and can stabilize the DFG-out conformation by forming π-π stacking interactions between the DFG Phe169 and Tyr35 of the glycine-rich loop, thus explaining the sensitive detection of this compound using both the method of EP 08 02 0341 and the present invention. The Type I binding mode of SB203580 was easily discriminated from Type II/III binders in a HTS format due to its more rapid binding and dissociation kinetics and because it did not show the same K_{d} time dependence as the DFG-out binder BIRB-796 (FIG. 7). Thus, the method of the present invention also allows easy preliminary assessments of inhibitor binding mode without requiring co-crystallization of detected ligands with the protein.

Dasatinib, a Type I inhibitor that binds to p38α with a reported K_{d} of 27 nM (Karaman et al., 2008) was not detected using glycine-rich loop-labelled p38α, suggesting that it adopts the expected/published binding mode observed in the DFG-in conformation of Abl (PDB code: 2GQG) and cSrc (PDB code: 3G5D). However, it was surprising to observe that the indole derivative Scios-469 and the 2-phenyl-substituted quinazoline RL40, both known to adopt the classical Type I binding mode and to contact the hinge region (Murali et al., 2007; Pierce et al., 2005) were detected using this approach. In the case of Scios-469, Applicants determined K_{d} value of 8.2±2.9 nM, which strongly agrees with the reported IC50 for this compound (˜9 nM) (Murali Dhar et al., 2007) and demonstrates that the method of the present invention is extremely sensitive to this ligand. Additionally, the VEGFR2 inhibitor CP547632 (CP-547632) was also detected and was found to have K_{d} of 99±/−13 nM. This compound is in clinical trials as an anticancer agent that acts by inhibiting angiogenesis and tumor growth mediated by VEGFR2 (Beebe et al., 2003). Although no crystal structure for CP547632 in complex with a kinase has been published to date, previously reported pharmacokinetic studies revealed that CP547632 inhibits VEGFR2 in an ATP-competitive manner (Beebe et al., 2003). Real-time kinetic measurements of RL40, Scios-469, and CP547632 show rapid binding (<2 s) of all three compounds to p38α, which is consistent with the expected Type I binding mode. However, only CP547632 could be characterized using p38α labelled at either the activation loop or the glycine-rich loop; only the latter detected RL40 and Scios-469. This suggests that the present invention has the added advantage of detecting certain Type I inhibitors that may induce unique conformational changes, most likely by making additional contacts to the acrylodan-labelled glycine-rich loop and altering its conformation.

With respect to the determined K_{d} values for detected ligands expected to bind to p38α, the affinities are in very close agreement with those determined and published elsewhere using other methods (Pargellis et al. 2003, EP 08 01 3340 and EP 08 02 0341). The close agreement between the K_{d} values and the kinetic trends reported using the P-loop labelled assay and other methods validates the assay system. Additionally, a Type II ligand known to not bind to p38α (imatinib) and a Type I inhibitor (dasatinib) that potently inhibits p38α (Karaman et al 2008), but does not interact with the DFG motif or the P-loop, were not detected by the assay and could be used as negative controls (FIG. 3a-right; FIG. 3b-right).

### EXAMPLE 6

### Crystal Structures of RL40, Scios-469 and CP547632 Confirm Movement of the P-Loop

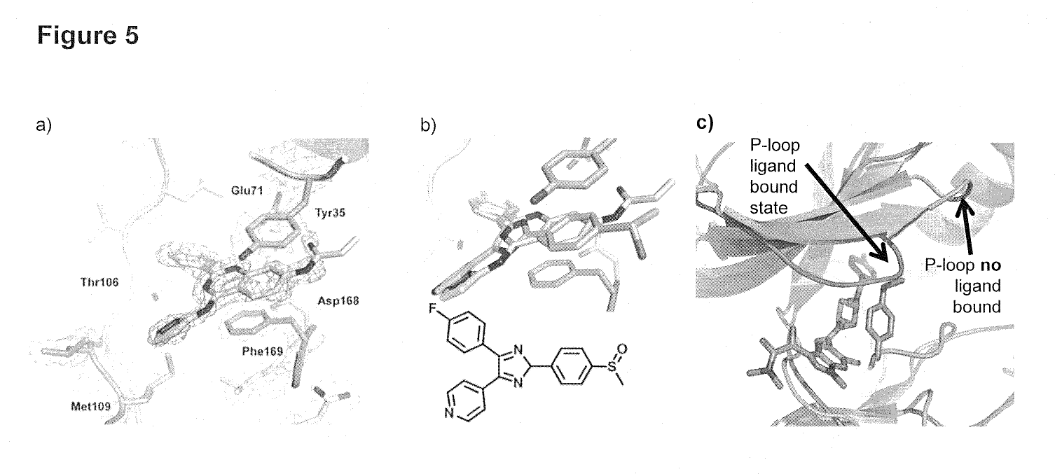

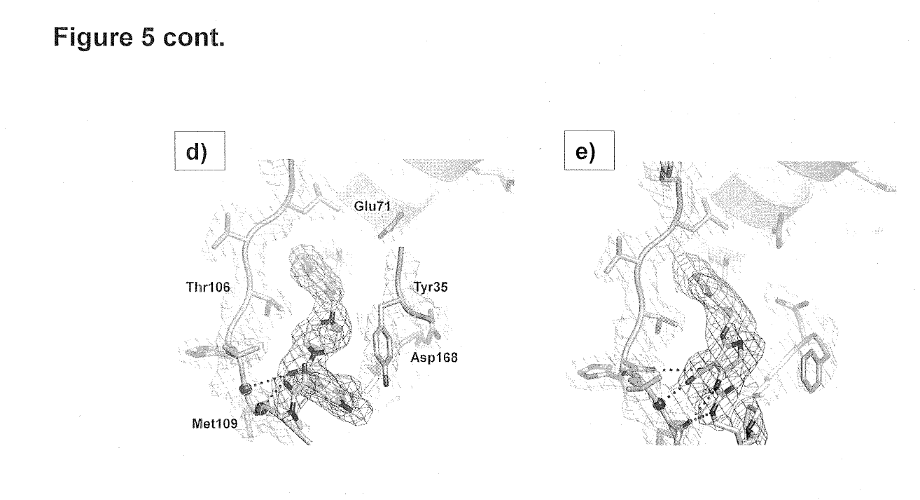

Applicants screened several compounds against P-loop labelled p38α and identified RL40, Scios-469 and CP547632 as being sensitively detected in the assay. To understand the structural details which explain the detection of these ligands, Applicants co-crystallized RL40, Scios-469 and CP547632 with wild-type p38α. Inhibitors were co-crystallized with unlabelled p38α. Briefly, protein inhibitor complexes were prepared by mixing 30 μL of p38α (10 mg/mL) with 0.3 μL of inhibitor (100 mM in DMSO) and incubating the mixture for 1-2 h on ice. Samples were centrifuged at 13 000 rpm for 5 min to remove excess inhibitor. Crystals were grown in 24-well crystallization plates using the hanging drop vapour diffusion method and by mixing 1.5 μL of protein-inhibitor solution with 0.5 μL of reservoir (100 mM MES pH 5.6-6.2, 20-30% PEG4000 and 50 mM n-octyl-β-D-glucopyranoside). The structure of RL40 in complex with p38α (FIG. 5A) reveals a unique and unexpected binding mode which is analogous to that observed in the structure of SB203580 reported previously (EP 08 01 3340 and EP 08 02 0341). The ligand interacts with the P-loop by forming a unique π-π stacking with the Phe of the DFG motif and thereby stabilizes the DFG-out conformation and provides a rational explanation for its detection in the assay. An overlay of this structure with that of the SB203580-p38α complex (FIG. 5B) reveals that features of both inhibitors nicely overlay and provides insight into future chemical modifications to improve affinity. However, unlike SB203580, compound RL40 does not contact the hinge region, and the attached acrylamide extends further in the direction of the position used to label the activation loop disclosed in EP 08 01 3340 and EP 08 02 0341. Therefore, activation loop labelling at this position may be incompatible with the binding mode of RL40 described here and might explain why it was previously not possible to detect and report the binding of RL40 to p38α. This unique binding mode differs significantly from those of previously reported structural analogues of RL40 in complex with GSK3 (Pierce et al., 2005) and calmodulin-dependent protein kinase 1D (PDB code: 2JC6), where the the inhibitor adopts a completely different orientation within the ATP binding site, amino-pyrazole moiety of the inhibitor strongly interacts with the hinge region via three hydrogen bonds and the kinase is found in the DFG-in conformation. The binding mode was unexpected since analogues of RL40 typically bind to the hinge region of kinases. These findings highlight the benefit of using P-loop labelled kinases to enrich for ligands which take advantage of this unique binding modes such as RL40 and SB203580.

Additionally, the P-loop labelled kinase assay strongly detected the binding of Scios-469. Applicants co-crystallized Scios-469 with wild-type p38α (FIG. 5C) and observed that the inhibitor forms hydrogen bonds to the hinge region, analogous to those previously reported for a close structural analogue (PDB code: 2QD9). The fluorophenyl moiety of Scios-469 extends beyond the gatekeeper residue and occupies the hydrophobic subpocket, an interaction that is known to increase the affinity of compounds for p38α (Lafont et al., 2007). Although the DFG motif is found in the active “in” conformation, the conformation of the glycine-rich loop is significantly altered in the case of p38α bound with Scios-469, thus explaining its sensitive detection using only the method of the present invention. The glycine-rich loop is folded over the inhibitor such that the side chain of Tyr35 partly shields the methyl-substituted piperazine ring and the chloro-substituted indole ring of Scios-469 from the solvent, thereby stabilizing inhibitor binding, most likely via hydrophobic interactions. As a consequence, the removal of ordered water molecules from this surface of the ligand results in the high-affinity binding of Scios-469 to p38α. Aside from detected all DFG-out binders, Scios-469 provides an example of how the P-loop labelled kinase assay can sensitively detect some Type I ligands which directly alter the conformation of the P-loop.

The crystal structure of CP547632 in complex with p38α (FIG. 5d) revealed that the kinase was stabilized in the DFG-out conformation with the inhibitor bound also to the hinge region (Type I). The carboxamide attached to the isothiazole of the inhibitor core forms two parallel hydrogen bonds to the hinge region (CO of His107 and NH of Met109). To the best of Applicants' knowledge, this represents a new hinge region binding motif. In addition, the two NH's of the urea moiety are pointing toward the backbone CO of Met109 and serve as hydrogen-bonding donors. The aliphatic linker of the solubilising pyrrolidine-butane moiety is surprisingly not pointing toward the solvent but rather folded inward toward the ATP pocket. The glycine-rich loop is relatively mobile in this complex and is partially not observed in the crystal structure. Therefore, the movement of the activation loop to its inactive conformation and its stabilization by CP547632 induces an upward movement of the acrylodan-labelled glycine-rich loop of p38α (see FIG. 1). This explains the sensitive detection of CP547632 with the method of the present invention as well as with the method in which the activation loop of p38α was labelled.

### EXAMPLE 7

### Real-Time and Endpoint Fluorescence Measurements Using ac-MKK7 Labelled on the Glycine-Rich Loop

All experiments were carried out as for p38α in FIG. 3 using only the kinase domain of MKK7 (SEQ ID NO: 2). The cysteine labelled is naturally present in the P-loop at position 147 (position 31 in SEQ ID NO:2). Cysteines at positions 218, 276 and 296 (positions 102, 160 and 180 of SEQ ID NO: 2) were replaced with serine (SEQ ID NO: 3).

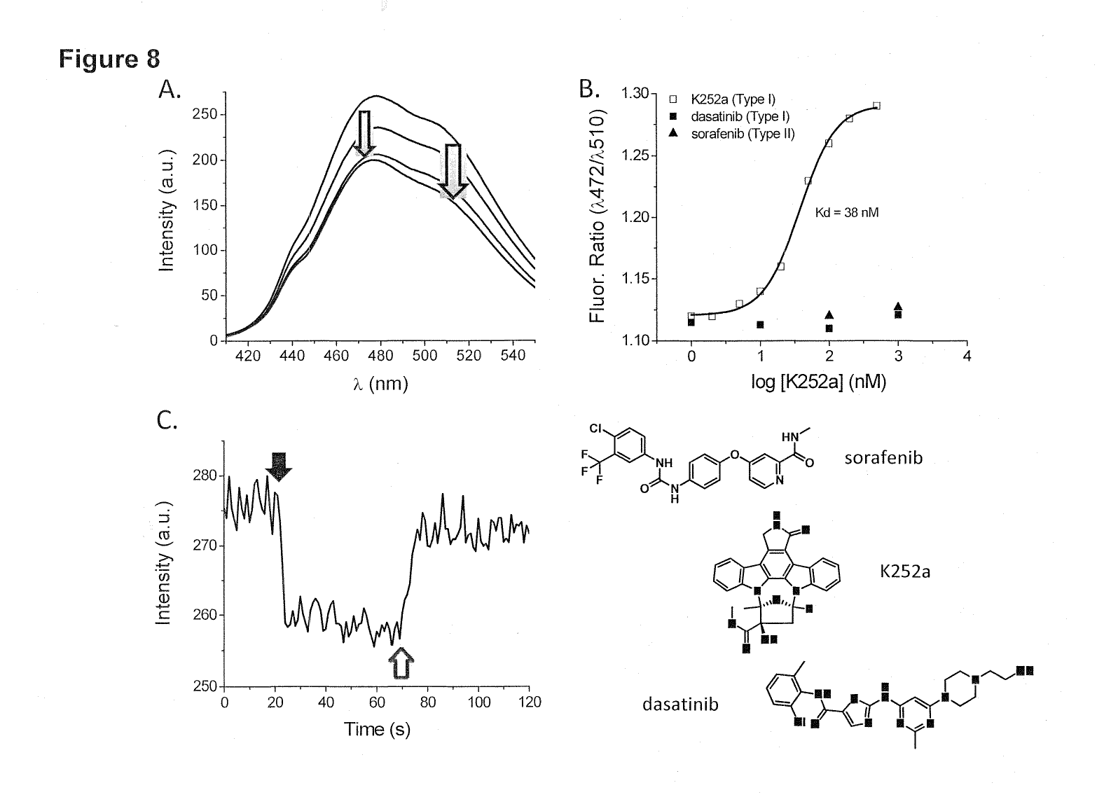

MKK7 represents an example of a kinase which may not be sensitive to the DFG-out conformation which is amenable to the binding of Type II and Type III inhibitors. Although the current invention demonstrates the ability of the assay to discriminate between ligands which bind to the DFG-out conformation (slower binding kinetics), it is also sensitive to many Type I inhibitors which interact with the P-loop directly and modify its conformation. According to the kinase profiling for several inhibitors against a panel of more than 300 kinases by Karaman et al. 2008, all of the closest homologues of MKK7 (MKK1-6, also known as MEK1-6), are not inhibited by Type II inhibitors with a K_{d} value <10 μM. However, they are inhibited strongly by staurosporine with K_{d} values in the range of 3.4-70 nM. Staurosporine and its closest derivatives potently inhibit >90% of all kinases in an ATP-competitive manner. MKK7 was not part of this kinase panel at the time, but is expected to exhibit similar inhibitor preference and profiles as its close homologues. Therefore, the K_{d} of a Type I inhibitor (K252a) to P-loop labelled MKK7 was measured. K252a is a promiscuous Type I inhibitor and is a close structural analogue of staurosporine. FIG. 8A shows that the binding of K252a induces a decrease in fluorescence intensity of the labelled protein and a detectable change in the ratiometric emission at two wavelengths (R=472 nm/510 nm). Using the endpoint methodology to directly determine K_{d}, the ratio of these emissions can be plotted against inhibitor concentration to obtain a K_{d} of 38 nM for K252a (FIG. 8B), which is in the correct range expected for these compounds. As negative controls, sorafenib was included, a Type II inhibitor, and was not detected up to 10 μM. These findings are in line with expected results for MKK7, which shows an insensitivity to the DFG-out conformation. To demonstrate that the assay response is due to movement of the P-loop in response to Type I inhibitor binding, dasatinib was also included as a negative control. Dasatinib is an ATP-competitive inhibitor or cSrc and Abl kinases, only inhibits MKK1 and MKK2 but with reported K_{d} values >1 (Karaman et al. 2008) and does not interact typically with the P-loop of kinases in any known crystal structure. Therefore, addition and detection of this Type I inhibitor was not expected for MKK7, which the data confirm up to 10 μM. FIG. 8C highlights the real-time kinetic measurements and detection of binding and dissociation of K252a. As in FIG. 3 for p38α, the fluorescence change which occurs with binding is reversible upon addition of excess unlabelled MKK7 to extract the ligand from the labelled kinase. Since K252a is a Type I inhibitor, the kinetics of these processes are fast, as for the Type I inhibitor SB203580 of p38α shown in FIG. 3B.

### REFERENCES

Adrian, F. J., Ding, Q., Sin, T., Velentza, A., Sloan, C., Liu, Y., Zhang, G., Hur, W., Ding, S., Manley, P., Mestan, J., Fabbro, D., Gray, N. S. Nat Chem Biol 2006, 2, 95-102.
Annis, D. A., Nazef, N., Chuang, C. C., Scott, M. P., and Nash, H. M. (2004) A general technique to rank protein-ligand binding affinities and determine allosteric versus direct binding site competition in compound mixtures, Journal of the American Chemical Society 126, 15495-15503.
R. T. Aimes, W. Hemmer, S. S. Taylor. Serine-53 at the tip of the glycine-rich loop of cAMP-dependent protein kinase: role in catalysis, P-site specificity, and interaction with inhibitors. Biochemistry 2000, 39, 8325.
B. Apsel, J. A. Blair, B. Gonzalez, T. M. Nazif, M. E. Feldman, B. Aizenstein, R. Hoffman, R. L. Williams, K. M. Shokat, Z. A. Knight, Nat Chem Biol 2008, 4, 691.
A. C. Backes, B. Zech, B. Felber, B. Klebl, G. Müller, Expert Opin Drug Discovery 2008, 3, 1409.
A. C. Backes, B. Zech, B. Felber, B. Klebl, G. Müller, Expert Opin Drug Discovery 2008, 3, 1427.
Beebe, J. S., Jani, J. P., Knauth, E., Goodwin, P., Higdon, C., Rossi, A. M., Emerson, E., Finkelstein, M., Floyd, E., Harriman, S., Atherton, J., Hillerman, S., Soderstrom, C., Kou, K., Gant, T., Noe, M. C., Foster, B., Rastinejad, F., Marx, M. A., Schaeffer, T., Whalen, P. M., Roberts, W. G. (2003). Pharmacological characterization of CP-547,632, a novel vascular endothelial growth factor receptor-2 tyrosine kinase inhibitor for cancer therapy. Cancer Res 63, 7301-9.
J. A. Blair, D. Rauh, C. Kung, C. H. Yun, Q. W. Fan, H. Rode, C. Zhang, M. J. Eck, W. A. Weiss, K. M. Shokat, Nat Chem Biol 2007, 3, 229.
Calleja, V., Laguerre, M., Parker, P. J., Larijani, B. PLoS Biol 2009, 7, 189-200.
R. A. Copeland, D. L. Pompliano, T. D. Meek, Nat Rev Drug Discov 2006, 5, 730.
Dar, A. C., Lopez, M. S., and Shokat, K. M., Small Molecule Recognition of c-Src via the Imatinib-Binding Conformation. Chem Biol 15 (10), 1015 (2008). DeLano, W. L., The PyMOL Molecular Graphics System. http://www.pymol.org (2002).
de Lorimier, R. M., Smith, J. J., Dwyer, M. A., Looger, L. L., Sali, K. M., Paavola, C. D., Rizk, S. S., Sadigov, S., Conrad, D. W., Loew, L., and Hellinga, H. W. (2002) Construction of a fluorescent biosensor family, Protein Sci 11, 2655-2675. Dumas, J., Hatoum-Mokdad, H., Sibley, R., Riedl, B., Scott, W. J., Monahan, M. K., Lowinger, T. B., Brennan, C., Natero, R., Turner, T., Johnson, J. S., Schoenleber, R., Bhargava, A., Wilhelm, S. M., Housley, T. J., Ranges, G. E., and Shrikhande, A. (2000) 1-Phenyl-5-pyrazolyl ureas: potent and selective p38 kinase inhibitors, Bioorganic & medicinal chemistry letters 10, 2051-2054.
Emsley, P. and Cowtan, K., Coot: model-building tools for molecular graphics. Acta Crystallogr D Biol Crystallogr 60 (Pt 12 Pt 1), 2126 (2004).
Eyers, P. A., Churchill, M. E., Maller, J. L. (2005). The Aurora A and Aurora B protein kinases: a single amino acid difference controls intrinsic activity and activation by TPX2. Cell Cycle 4, 784-9.
Fischmann, T., Smith, C., Mayhood, T., Myers, J., Reichert, P., Mannarino, A., Carr, D., Zhu, H., Wong, J., Yang, R. S., Le, H., Madison, V., Biochemistry 2009.
M. J. Garnett, R. Marais, Cancer Cell 2004, 6, 313.
Gauglitz, G. and Vo-Dinh, T. (2003). Handbook of spectroscopy. Wiley-VCH.
Getlik, M., Grutter, C., Simard, J. R., Kluter, S., Rabiller, M., Rode, H. B., Robubi, A., Rauh, D. (2009). Hybrid compound design to overcome the gatekeeper T338M mutation in cSrc. J Med Chem 52, 3915-26.
Gschwind, A., Fischer, O. M. and Ullrich, A., The discovery of receptor tyrosine kinases: targets for cancer therapy. Nat Rev Cancer 4 (5), 361 (2004).
S. K. Hanks, T. Hunter, FASEB J 1995, 9, 576.
Hibbs, R. E., Talley, T. T., and Taylor, P. (2004) Acrylodan-conjugated cysteine side chains reveal conformational state and ligand site locations of the acetylcholine-binding protein, The Journal of biological chemistry 279, 28483-28491.
Johnson, L. N., Noble, M. E. M. and Owen, D. J. (1996) Active and inactive protein kinases: structural basis for regulation. Cell 85, 149-158. Johnson, L. N. and Lewis, R. E. (2001). Structural basis for control by phosphorylation. Chem Rev. 2001 August; 101(8):2209-42.
Kabsch, W., Automatic processing of rotation diffraction data from crystals of initially unknown symmetry and cell constants. J. Appl. Cryst. 26, 795 (1993).
Karaman, M. W., Herrgard, S., Treiber, D. K., Gallant, P., Atteridge, C. E., Campbell, B. T., Chan, K. W., Ciceri, P., Davis, M. I., Edeen, P. T., Faraoni, R., Floyd, M., Hunt, J. P., Lockhart, D. J., Milanov, Z. V., Morrison, M. J., Pallares, G., Patel, H. K., Pritchard, S., Wodicka, L. M., Zarrinkar P. P. (2008). A quantitative analysis of kinase inhibitor selectivity. Nat Biotechnol 26, 127-32.
Kirkland, L. O., McInnes, C. Biochem Pharmacol 2009.
Kluter, S., Grutter, C., Naqvi, T., Rabiller, M., Simard, J. R., Pawar, V., Getlik, M., Rauh, D. (2009). Displacement assay for the detection of stabilizers of inactive kinase conformations. J Med Chem
Knighton, D. R., Zheng, J. H., Ten Eyck, L. F., Xuong, N. H, Taylor, S. S., Sowadski, J. M. (1991). Structure of a peptide inhibitor bound to the catalytic subunit of cyclic adenosine monophosphate-dependent protein kinase. Science 253, 414-20.
Lafont, V., Armstrong, A. A., Ohtaka, H., Kiso, Y., Mario Amzel, L., Freire, E. (2007). Chem Biol Drug Des 69, 413-22.
Lakowicz, J. R. (1999). Principles of Fluorescence Spectroscopy. Kluwer Academic/Plenum Publishers
Laskowski, R. A., MacArthur, M. W., Moss, D. S., and Thornton, J. M., PROCHECK: a program to check the stereochemical quality of protein structures. J Appl Cryst 26, 283 (1993).
Larkin, M. A. et al., Clustal W and Clustal X version 2.0. Bioinformatics 23 (21), 2947 (2007).
Levinson, N. M. et al., A Src-like inactive conformation in the abl tyrosine kinase domain. PLoS Biol 4 (5), e144 (2006).
Liu, Y. and Gray, N. S., Rational design of inhibitors that bind to inactive kinase conformations. Nat Chem Biol 2 (7), 358 (2006).
Liu, C. C. and Schultz, P. G. (2010). Adding new chemistries to the genetic code. Annu Rev Biochem 79, 15.1-15.32.
M. Mapelli, L. Massimiliano, C. Crovace, M. A. Seeliger, L. H. Tsai, L. Meijer, A. Musacchio, J Med Chem 2005, 48, 671.
Michalczyk, A. et al., Structural insights into how irreversible inhibitors can overcome drug resistance in EGFR. Bioorg Med Chem 16 (7), 3482 (2008).
Murali Dhar, T. G., Wrobleski, S. T., Lin, S., Furch, J. A., Nirschl, D. S., Fan, Y., Todderuf, G., Pitt, S., Doweyko, A. M., Sack, J. S., Mathur, A., McKinnon, M., Barrish, J. C., Dodd, J. H., Schieven, G. L., Keftheris, K. (2007). Synthesis and SAR of p38alpha MAP kinase inhibitors based on heterobicyclic scaffolds. Bioorg Med Chem Lett 17, 5019-24.
Murshudov, G. N., Vagin, A. A., and Dodson, E. J., Refinement of macromolecular structures by the maximum-likelihood method. Acta Crystallogr D Biol Crystallogr 53 (Pt 3), 240 (1997).
B. Nagar, W. G. Bornmann, P. Pellicena, T. Schindler, D. R. Veach, W. T. Miller, B. Clarkson, J. Kuriyan, Cancer Res 2002, 62, 4236.
Nienaber, V. L. et al., Discovering novel ligands for macromolecules using X-ray crystallographic screening. Nature biotechnology 18 (10), 1105 (2000).
Pargellis, C., Tong, L., Churchill, L., Cirillo, P. F., Gilmore, T., Graham, A. G., Grob, P. M., Hickey, E. R., Moss, N., Pav, S., and Regan, J. (2002) Inhibition of p38 MAP kinase by utilizing a novel allosteric binding site, Nature structural biology 9, 268-272.
Patel, S. B., Cameron, P. M., O'Keefe, S. J., Frantz-Wattley, B., Thompson, J., O'Neill, E. A., Tennis, T., Liu, L., Becker, J. W., Scapin, G. Acta Crystallogr D Biol Crystallogr 2009, 65, 777-85.
Pawar, V. G., Sos, M. L., Rode, H. B., Rabiller, M., Heynck, S., van Otterlo, W. A. L., Thomas, R. K., Rauh, D. (2010), Synthesis and biological evaluation of 4-anilinoquinolines as potent inhibitors of epidermal growth factor receptor. J Med Chem 53(7), 2892-901.
W. A Pearson, Rapid and Sensitive Sequence Comparison with FASTP and FASTA, in Methods in Enzymology, ed. R. Doolittle Academic Press, San Diego) 183(1990)63-98.
Pierce A C, ter Haar E, Binch H M, Kay D P, Patel S R, Li P. J Med Chem. 2005 Feb. 24; 48(4):1278-81.
Read, R. J., Pushing the boundaries of molecular replacement with maximum likelihood. Acta Crystallogr D 57 (Pt 10), 1373 (2001).
Reich, M. et al., Gene Pattern 2.0. Nat Genet 38 (5), 500 (2006).
Regan, J., Breitfelder, S., Cirillo, P., Gilmore, T., Graham, A. G., Hickey, E., Klaus, B., Madwed, J., Moriak, M., Moss, N., Pargellis, C., Pav, S., Proto, A., Swinamer, A., Tong, L., and Torcellini, C. (2002) Pyrazole urea-based inhibitors of p38 MAP kinase: from lead compound to clinical candidate, Journal of medicinal chemistry 45, 2994-3008.
Regan, J., Pargellis, C. A., Cirillo, P. F., Gilmore, T., Hickey, E. R., Peet, G. W., Proto, A., Swinamer, A., and Moss, N. (2003) The kinetics of binding to p38MAP kinase by analogues of BIRB 796, Bioorganic & medicinal chemistry letters 13, 3101-3104.
Rendell, D. (1987). Fluorescence and Phosphorescence. Crown
Richieri, G. V., Ogata, R. T., and Kleinfeld, A. M. (1999) The measurement of free fatty acid concentration with the fluorescent probe ADIFAB: a practical guide for the use of the ADIFAB probe, Molecular and cellular biochemistry 192, 87-94. M. Saraste, P. R. Sibbald, A. Wittinghofer, Trends Biochem Sci 1990, 15, 430.
Schuttelkopf, A. W. and van Aalten, D. M., PRODRG: a tool for high-throughput crystallography of protein-ligand complexes. Acta Crystallogr D Biol Crystallogr 60 (Pt 8), 1355 (2004).
Sharma, A. and Schulman, S. G. (1999). Introduction to Fluorescence Spectroscopy. Wiley interscience. Sullivan, J. E., Holdgate, G. A., Campbell, D., Timms, D., Gerhardt, S., Breed, J., Breeze, A. L., Bermingham, A., Pauptit, R. A., Norman, R. A., Embrey, K. J., Read, J., VanScyoc, W. S., and Ward, W. H. (2005) Prevention of MKK6-dependent activation by binding to p38alpha MAP kinase, Biochemistry 44, 16475-16490.
M. A. Seeliger, B. Nagar, F. Frank, X. Cao, M. N. Henderson, J. Kuriyan, Structure 2007, 15, 299.
Shuker, S. B., Hajduk, P. J., Meadows, R. P., and Fesik, S. W., Discovering high-affinity ligands for proteins: SAR by NMR. Science 274 (5292), 1531 (1996).
J. R. Simard, S. Klüter, C. Grütter, M. Getlik, M. Rabiller, H. B. Rode, D. Rauh, Nat Chem Biol 2009, 5, 394-396.
Simard, J. R., Getlik, M., Grutter, C., Pawar, V., Wulfert, S., Rabiller, M., Rauh, D. (2009). Development of a fluorescent-tagged kinase assay system for the detection and characterization of allosteric kinase inhibitors. J Am Chem Soc 131, 13286-13296.
Simard, J. R., Pawar, V., Aust, B., Wolf, A., Rabiller, M., Wulfert, S., Robubi, A., Kluter, S., Ottmann, C., Rauh, D. (2009). High-throughput screening to identify inhibitors which stabilize inactive kinase conformations in p38alpha. J Am Chem Soc
Sos, M. L., Rode, H. B., Heynck, S., Peifer, M., Fischer, F., Klüter, S., Pawar, V. G., Reuter, C., Heuckmann, J., Weiss, J., Ruddigkeit, L., Rabiller, M., Koker, M., Simard, J. R., Getlik, M., Yuza, Y., Chen, T., Greulich, H., Meyerson, M., Thomas, R. K., Rauh, D. (2010). Chemogenomic profiling provides insights into the limited activity of irreversible EGFR Inhibitors in tumor cells expressing the T790M EGFR resistance mutation. Cancer Research 70(3):868-74.
Tamayo, N, Liao, L., Goldberg, M., Powers, D., Tudor, Y-Y, Yu, V., Wong, L. M., Henkle, B., Middleton, S., Syed, R., Harvey, T., Jang, G., Hungate, R. and Celia Dominguez (2005). Design and synthesis of potent pyridazine inhibitors of p38 MAP kinase, Bioorganic & Medicinal Chemistry Letters 15:2409-2413.
Tecle, H., Feru, F., Liu, H., Kuhn, C., Rennie, G., Morris, M., Shao, J., Cheng, A. C., Gikunju, D., Miret, J., Coli, R., Xi, S. H., Clugston, S. L., Low, S., Kazmirski, S., Ding, Y. H., Cao, Q., Johnson, T. L., Deshmukh, G. D., DiNitto, J. P., Wu, J. C.; English, J. M. (2009). The design, synthesis and potential utility of fluorescence probes that target DFG-out conformation of p38alpha for high throughput screening binding assay. Chem Biol Drug Des 74, 547-59.
Taylor, S. S. and Radzio-Andzelm, E. (1994) Three protein kinase structures define a common motif. Structure 2, 345-355; 43. Vogtherr, M., Saxena, K., Hoelder, S., Grimme, S., Betz, M., Schieborr, U., Pescatore, B., Robin, M., Delarbre, L., Langer, T., Wendt, K. U., and Schwalbe, H. (2006) NMR characterization of kinase p38 dynamics in free and ligand-bound forms, Angewandte Chemie (International ed 45, 993-997. Yeatman, T. J., A renaissance for SRC. Nat Rev Cancer 4 (6), 470 (2004).
Tokarski, J. S., Newitt, J., Chang, C. Y. J., Cheng, J. D., Wittekind, M., Kiefer, S. E., Kish, K., Lee, F. Y. F., Borzilerri, R., Lombardo, L. J., Xie, D., Zhang, Y., Klei, H. E. (2006) CANCER RES. 66: 5790-5797.
Wan, P. T., Garnett, M. J., Roe, S. M., Lee, S., Niculescu-Duvaz, D., Good, V. M., Jones, C. M., Marshall, C. J., Springer, C. J., Barford, D., Marais, R., Cell 2004, 116, 855.
Wang, X, Destrument, A, Tournier, C. (2007). Physiological roles of MKK4 and MKK7: Insights from animal models. Biochimica et Biophysica Acta 1773 (2007), 1349-1357.
Wong, L., Lieser, S. A., Miyashita, O., Miller, M., Tasken, K., Onuchic, J. N., Adams, J. A., V. L. Woods, Jr., P. A. Jennings, J Mol Biol 2005, 351, 131.
Yem, A. W., Epps, D. E., Mathews, W. R., Guido, D. M., Richard, K. A., Staite, N. D., and Deibel, M. R., Jr. (1992) Site-specific chemical modification of interleukin-1 beta by acrylodan at cysteine 8 and lysine 103, The Journal of biological chemistry 267, 3122-3128.
J. Zhang, P. L. Yang, N. S. Gray, Nat. Rev. Cancer 2009, 9, 28.

The invention is further described by the following numbered paragraphs:

1. A kinase labelled at an amino acid naturally present or introduced in the P-loop of said kinase, wherein said labelling is effected at a free thiol or amino group of said amino acid and said label is

(a) a thiol- or amino-reactive fluorophore sensitive to polarity changes in its environment; or

(b) a thiol-reactive spin label, an isotope or an isotope-enriched thiol- or amino-reactive label;

such that said fluorophore, spin label, isotope or isotope-enriched label does not inhibit the catalytic activity and does not interfere with the stability of the kinase.

2. The kinase of paragraph 1, which is a serine/threonine or tyrosine kinase.

3. The kinase of paragraph 1 or 2, which is MEK kinase, CSK, an Aurora kinase, GSK-3beta, cSrc, EGFR, Abl, DDR1, AKT, LCK, a CDK, p38α or another MAPK.

4. The kinase of any one of paragraphs 1 to 3, wherein the amino acid labelled is cysteine, lysine, arginine or histidine.

5. The kinase of any one of paragraphs 1 to 4, wherein one or more solvent-exposed cysteines present outside the P-loop are deleted or replaced.

6. The kinase of any one of paragraphs 3 to 5, which is p38α and wherein a cysteine to be labelled is introduced at position 35 of SEQ ID NO: 1 and preferably wherein the cysteines at position 119 and 162 of SEQ ID NO: 1 are replaced with another amino acid.

7. The kinase of any one of paragraphs 1 to 6, wherein the thiol- or amino-reactive fluorophore is a di-substituted naphthalene compound, a coumarin-based compound, a benzoxadiazole-based compound, a dapoxyl-based compound, a biocytin-based compound, a fluorescein, a sulfonated rhodamine-based compound, Atto fluorophores or Lucifer Yellow or derivatives thereof which exhibit a sensitivity to environmental changes.

8. The kinase of any one of paragraphs 1 to 6, wherein the thiol-reactive spin-label is a nitroxide radical.

9. A method of screening for kinase inhibitors comprising

(a) providing a kinase according to any one of paragraphs 1 to 8

(b) contacting said fluorescently or spin-labelled or isotope-labelled kinase with a candidate inhibitor;

(c) recording the fluorescence emission signal at one or more wavelengths or a spectrum of said fluorescently labelled kinase of step (a) and step (b) upon excitation; or

(c)′ recording the electron paramagnetic resonance (EPR) or nuclear magnetic resonance (NMR) spectra of said spin-labelled or isotope-labelled kinase of step (a) and step (b); and

(d) comparing the fluorescence emission signal at one or more wavelengths or the spectra recorded in step (c) or the EPR or NMR spectra recorded in step (c)′;

wherein a difference in the fluorescence intensity at at least one wavelength, preferably at the emission maximum, and/or a shift in the fluorescence emission wavelength in the spectra of said fluorescently labelled kinase obtained in step (c), or an alteration in the EPR or NMR spectra of said spin-labelled or isotope-labelled kinase obtained in step (c)′ indicates that the candidate inhibitor is a kinase inhibitor.

10. A method of determining the kinetics of ligand binding and/or of association or dissociation of a kinase inhibitor comprising

(a) contacting a fluorescently labelled kinase according to any one of paragraphs 1 to 8 with different concentrations of an inhibitor; or

(a)′ contacting a fluorescently labelled kinase according to any one of paragraphs 1 to 8 bound to an inhibitor with different concentrations of unlabelled kinase;

(b) recording the fluorescence emission signal at one or more wavelengths or a spectrum of said fluorescently labelled kinase for each concentration upon excitation;

(c) determining the rate constant for each concentration from the fluorescence emission signals at one or more wavelengths or the spectra recorded in step (b); or

(c1) determining the K_{d} from the fluorescence emission signal at one or more wavelengths or the spectra recorded in step (b) for each concentration of inhibitor; or

(c2) determining the Kₐ from the fluorescence emission signal at one or more wavelengths or the spectra recorded in step (b) for each concentration of unlabelled kinase;

(d) directly determining the kₒₙ and/or extrapolating the k_{off} from the rate constants determined in step (c) from the signals or spectra for the different concentrations of inhibitor obtained in step (b); or

(d)′ directly determining the k_{off} and/or extrapolating the kₒₙ from the rate constants determined in step (c) from the signals or spectra for the different concentrations of unlabelled kinase obtained in step (b); and

(e) optionally calculating the K_{d} and/or Ka from kₒₙ and k_{off} obtained in step (d) or (d)′.

11. A method of determining the dissociation or association of a kinase inhibitor comprising

(a) contacting a spin-labelled or isotope-labelled kinase according to any one of paragraphs 1 to 8 with different concentrations of an inhibitor; or

(a)′ contacting a spin-labelled or isotope-labelled kinase according to any one of paragraphs 1 to 8 bound to an inhibitor with different concentrations of unlabelled kinase;

(b) recording the EPR or NMR spectrum of said spin-labelled or isotope-labelled kinase for each concentration of inhibitor and/or unlabelled kinase; and

(c) determining the K_{d} from the EPR or NMR spectra recorded in step (b) for the different concentrations of inhibitor; or

(c)′ determining the Kₐ from the EPR or NMR spectra recorded in step (b) for the different concentrations of unlabelled kinase.

12. A method of generating a mutated kinase suitable for the screening of kinase inhibitors comprising

(a) replacing solvent exposed amino acids having a free thiol or amino group, if any, present in a kinase of interest outside the P-loop and/or amino acids having a free thiol or amino group at an unsuitable position within the P-loop with an amino acid not having a free thiol or amino group;

(b) mutating an amino acid in the P-loop of said kinase of interest to an amino acid having a free thiol or amino group if no amino acid having a free thiol or amino group is present in the P-loop;

(c) labelling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity of the kinase and/or does not interfere with the stability of the kinase;

(d) contacting the kinase obtained in step (c) with a known inhibitor of said kinase;

(e) recording the fluorescence emission signal at one or more wavelengths or a spectrum of said fluorescently labelled kinase of step (c) and (d) upon excitation; or

(e)′ recording the EPR or NMR spectra of said spin-labelled kinase of step (c) and (d); and

comparing the fluorescence emission spectra recorded in step (e) or the EPR or NMR spectra recorded in step (e)′;

wherein a difference in the fluorescence intensity at at least one wavelength, preferably at the emission maximum, and/or a shift in the fluorescence emission wavelength in the spectra of said fluorescently labelled kinase obtained in step (e), or an alteration in the EPR or NMR spectra of said spin-labelled or isotope-labelled kinase obtained in step (e)′ indicates that the kinase is suitable for the screening for kinase inhibitors.

13. The method of any one of paragraphs 9 to 12, wherein the kinase inhibitor binds either partially or fully to the allosteric site adjacent to the ATP binding site of the kinase.

14. A method for identifying a kinase inhibitor binding either partially or fully to the allosteric site adjacent to the ATP binding site of a kinase comprising

(a) screening for an inhibitor according to the method of paragraph 10; and

(b) determining the rate constant of an inhibitor identified in step (a);

wherein a rate constant of <0.140 s⁻¹ determined in step (b) indicates that the kinase inhibitor identified binds either partially or fully to the allosteric site adjacent to the ATP binding site of the kinase.

15. The kinase of any one of paragraphs 1 to 8 or the method of any one of paragraphs 9 to 14, wherein the kinase is labelled at a cysteine naturally present or introduced in the P-loop.

16. The method of any one of paragraphs 9 or 12 to 15, further comprising optimizing the pharmacological properties of a compound identified as inhibitor of said kinase.

17. The method of paragraph 16, wherein the optimization comprises modifying an inhibitor identified as inhibitor of said kinase to achieve:

a) modified spectrum of activity, organ specificity, and/or

b) improved potency, and/or

c) decreased toxicity (improved therapeutic index), and/or

d) decreased side effects, and/or

e) modified onset of therapeutic action, duration of effect, and/or

f) modified pharmacokinetic parameters (absorption, distribution, metabolism and excretion), and/or

g) modified physico-chemical parameters (solubility, hygroscopicity, color, taste, odor, stability, state), and/or

h) improved general specificity, organ/tissue specificity, and/or

i) optimized application form and route

by

a. esterification of carboxyl groups, or

b. esterification of hydroxyl groups with carboxylic acids, or

c. esterification of hydroxyl groups to, e.g. phosphates, pyrophosphates or sulfates or hemi-succinates, or

d. formation of pharmaceutically acceptable salts, or

e. formation of pharmaceutically acceptable complexes, or

f. synthesis of pharmacologically active polymers, or

g. introduction of hydrophilic moieties, or

h. introduction/exchange of substituents on aromates or side chains, change of substituent pattern, or

i. modification by introduction of isosteric or bioisosteric moieties, or

j. synthesis of homologous compounds, or

k. introduction of branched side chains, or

l. conversion of alkyl substituents to cyclic analogues, or

m. derivatization of hydroxyl groups to ketales, acetales, or

n. N-acetylation to amides, phenylcarbamates, or

o. synthesis of Mannich bases, imines, or

p. transformation of ketones or aldehydes to Schiff's bases, oximes, acetales, ketales, enolesters, oxazolidines, thiazolidines

or combinations thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A kinase labelled at an amino acid naturally present or introduced in the P-loop of said kinase, wherein said labelling is effected at a free thiol or amino group of said amino acid and said label is
(a) a thiol- or amino-reactive fluorophore sensitive to polarity changes in its environment; or
(b) a thiol-reactive spin label, an isotope or an isotope-enriched thiol- or amino-reactive label;
such that said fluorophore, spin label, isotope or isotope-enriched label does not inhibit the catalytic activity and does not interfere with the stability of the kinase.

2. The kinase of claim 1, which is a serine/threonine or tyrosine kinase.

3. The kinase of claim 1, which is MEK kinase, CSK, an Aurora kinase, GSK-3beta, cSrc, EGFR, Abl, DDR1, AKT, LCK, a CDK, p38α or another MAPK.

4. The kinase of claim 1, wherein the amino acid labelled is cysteine, lysine, arginine or histidine.

5. The kinase of claim 1, wherein one or more solvent-exposed cysteines present outside the P-loop are deleted or replaced.

6. The kinase of claim 3, which is p38α and wherein a cysteine to be labelled is introduced at position 35 of SEQ ID NO: 1 and preferably wherein the cysteines at position 119 and 162 of SEQ ID NO: 1 are replaced with another amino acid.

7. The kinase of claim 1, wherein the thiol- or amino-reactive fluorophore is a di-substituted naphthalene compound, a coumarin-based compound, a benzoxadiazole-based compound, a dapoxyl-based compound, a biocytin-based compound, a fluorescein, a sulfonated rhodamine-based compound, Atto fluorophores or Lucifer Yellow or derivatives thereof which exhibit a sensitivity to environmental changes.

8. The kinase of claim 1, wherein the thiol-reactive spin-label is a nitroxide radical.

9. A method of screening for kinase inhibitors comprising
(a) providing a kinase according to claim 1;
(b) contacting said fluorescently or spin-labelled or isotope-labelled kinase with a candidate inhibitor;
(c) recording the fluorescence emission signal at one or more wavelengths or a spectrum of said fluorescently labelled kinase of step (a) and step (b) upon excitation; or
(c)′ recording the electron paramagnetic resonance (EPR) or nuclear magnetic resonance (NMR) spectra of said spin-labelled or isotope-labelled kinase of step (a) and step (b); and
(d) comparing the fluorescence emission signal at one or more wavelengths or the spectra recorded in step (c) or the EPR or NMR spectra recorded in step (c)′;
wherein a difference in the fluorescence intensity at at least one wavelength, preferably at the emission maximum, and/or a shift in the fluorescence emission wavelength in the spectra of said fluorescently labelled kinase obtained in step (c), or an alteration in the EPR or NMR spectra of said spin-labelled or isotope-labelled kinase obtained in step (c)′ indicates that the candidate inhibitor is a kinase inhibitor.

10. A method of determining the kinetics of ligand binding and/or of association or dissociation of a kinase inhibitor comprising
(a) contacting a fluorescently labelled kinase according to claim 1 with different concentrations of an inhibitor; or
(a)′ contacting a fluorescently labelled kinase according to claim 1 bound to an inhibitor with different concentrations of unlabelled kinase;
(b) recording the fluorescence emission signal at one or more wavelengths or a spectrum of said fluorescently labelled kinase for each concentration upon excitation;
(c) determining the rate constant for each concentration from the fluorescence emission signals at one or more wavelengths or the spectra recorded in step (b); or
(c1) determining the K_{d} from the fluorescence emission signal at one or more wavelengths or the spectra recorded in step (b) for each concentration of inhibitor; or
(c2) determining the Kₐ from the fluorescence emission signal at one or more wavelengths or the spectra recorded in step (b) for each concentration of unlabelled kinase;
(d) directly determining the kₒₙ and/or extrapolating the k_{off} from the rate constants determined in step (c) from the signals or spectra for the different concentrations of inhibitor obtained in step (b); or
(d)′ directly determining the k_{off} and/or extrapolating the kₒₙ from the rate constants determined in step (c) from the signals or spectra for the different concentrations of unlabelled kinase obtained in step (b); and
(e) optionally calculating the K_{d} and/or Ka from kₒₙ and k_{off} obtained in step (d) or (d)′.

11. A method of determining the dissociation or association of a kinase inhibitor comprising
(a) contacting a spin-labelled or isotope-labelled kinase according to claim 1 with different concentrations of an inhibitor; or
(a)′ contacting a spin-labelled or isotope-labelled kinase according to claim 1 bound to an inhibitor with different concentrations of unlabelled kinase;
(b) recording the EPR or NMR spectrum of said spin-labelled or isotope-labelled kinase for each concentration of inhibitor and/or unlabelled kinase; and
(c) determining the K_{d} from the EPR or NMR spectra recorded in step (b) for the different concentrations of inhibitor; or
(c)′ determining the Kₐ from the EPR or NMR spectra recorded in step (b) for the different concentrations of unlabelled kinase.

12. A method of generating a mutated kinase suitable for the screening of kinase inhibitors comprising
(a) replacing solvent exposed amino acids having a free thiol or amino group, if any, present in a kinase of interest outside the P-loop and/or amino acids having a free thiol or amino group at an unsuitable position within the P-loop with an amino acid not having a free thiol or amino group;
(b) mutating an amino acid in the P-loop of said kinase of interest to an amino acid having a free thiol or amino group if no amino acid having a free thiol or amino group is present in the P-loop;
(c) labelling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity of the kinase and/or does not interfere with the stability of the kinase;
(d) contacting the kinase obtained in step (c) with a known inhibitor of said kinase;
(e) recording the fluorescence emission signal at one or more wavelengths or a spectrum of said fluorescently labelled kinase of step (c) and (d) upon excitation; or
(e)′ recording the EPR or NMR spectra of said spin-labelled kinase of step (c) and (d); and
(f) comparing the fluorescence emission spectra recorded in step (e) or the EPR or NMR spectra recorded in step (e)′;
wherein a difference in the fluorescence intensity at at least one wavelength, preferably at the emission maximum, and/or a shift in the fluorescence emission wavelength in the spectra of said fluorescently labelled kinase obtained in step (e), or an alteration in the EPR or NMR spectra of said spin-labelled or isotope-labelled kinase obtained in step (e)′ indicates that the kinase is suitable for the screening for kinase inhibitors.

13. The method of claim 9, wherein the kinase inhibitor binds either partially or fully to the allosteric site adjacent to the ATP binding site of the kinase.

14. A method for identifying a kinase inhibitor binding either partially or fully to the allosteric site adjacent to the ATP binding site of a kinase comprising
(a) screening for an inhibitor according to the method of claim 10; and
(b) determining the rate constant of an inhibitor identified in step (a);
wherein a rate constant of <0.140 s⁻¹ determined in step (b) indicates that the kinase inhibitor identified binds either partially or fully to the allosteric site adjacent to the ATP binding site of the kinase.

15. The kinase of claim 1 or the method of claim 9, wherein the kinase is labelled at a cysteine naturally present or introduced in the P-loop.

16. The method of claim 9, further comprising optimizing the pharmacological properties of a compound identified as inhibitor of said kinase.

17. The method of claim 16, wherein the optimization comprises modifying an inhibitor identified as inhibitor of said kinase to achieve:
a) modified spectrum of activity, organ specificity, and/or
b) improved potency, and/or
c) decreased toxicity (improved therapeutic index), and/or
d) decreased side effects, and/or
e) modified onset of therapeutic action, duration of effect, and/or
f) modified pharmacokinetic parameters (absorption, distribution, metabolism and excretion), and/or
g) modified physico-chemical parameters (solubility, hygroscopicity, color, taste, odor, stability, state), and/or
h) improved general specificity, organ/tissue specificity, and/or
i) optimized application form and route
by
a. esterification of carboxyl groups, or
b. esterification of hydroxyl groups with carboxylic acids, or
c. esterification of hydroxyl groups to, e.g. phosphates, pyrophosphates or sulfates or hemi-succinates, or
d. formation of pharmaceutically acceptable salts, or
e. formation of pharmaceutically acceptable complexes, or
f. synthesis of pharmacologically active polymers, or
g. introduction of hydrophilic moieties, or
h. introduction/exchange of substituents on aromates or side chains, change of substituent pattern, or
i. modification by introduction of isosteric or bioisosteric moieties, or
j. synthesis of homologous compounds, or
k. introduction of branched side chains, or
l. conversion of alkyl substituents to cyclic analogues, or
m. derivatization of hydroxyl groups to ketales, acetales, or
n. N-acetylation to amides, phenylcarbamates, or
o. synthesis of Mannich bases, imines, or
p. transformation of ketones or aldehydes to Schiff's bases, oximes, acetales, ketales, enolesters, oxazolidines, thiazolidines
or combinations thereof.
